(19) United States
(12) Patent Application Publication
(10) Pub. No.: US 2023/0285258 A1
(43) Pub. Date: Sep. 14, 2023

(54) A SUNSCREEN FORMULATION

(71) Applicant: Yimin Sun, Zhaoqing, Guangdong (CN)

(72) Inventor: Yimin Sun, Zhaoqing, Guangdong (CN)

(21) Appl. No.: 16/971,675

(22) PCT Filed: Sep. 10, 2019
(86) PCT No.: PCT/CN2019/105082

§ 371 Date: Aug. 20, 2020

(51) Int. Cl. A61K 8/49; A61K 8/36; A61Q 17/04; A61K 8/41; A61K 8/81

(52) U.S. Cl. CPC A61K 8/4966; A61K 8/36; A61K 8/411; A61K 8/8111; A61Q 17/04

(57) ABSTRACT

The present invention relates to the synthesis of new classes of sunscreen agents, the usage of such sunscreen agents to prepare sunscreen formulation materials aiming at reduction of sunscreen agents’ skin penetration. The whole purpose of this invention is to improve the biosafety of sunscreen products via our novel sunscreen materials and formulation technology to reduce or to eliminate skin penetration of sunscreen agents. The technology of this invention can be applied to cosmetics, pharmaceutical or insects’ repellant products.

### BACKGROUND OF INVENTION

### 1. Field of Invention

The present invention relates to the synthesis of UV rays blocker, the synthesis of UV rays blocker sunscreen formulation materials and usage of such materials to prepare sunscreen formulations with minimized of zero skin penetration of UV rays blocker for protection of human skin from solar UV radiation.

### 2. Description of Prior Art

Since the 1930s sunscreen has been used to prevent the maladies inflicted by the sun. There are several types of electromagnetic radiation emitted by the sun. Ultraviolet (UV) radiation is one type. Based on the characteristics of the UV rays interacted with human skin, it was divided into three bands based on wavelength: UVA rays (320-400 nm), UVB rays (290-320 nm) and UVC rays (100-290 nm). UVA radiation consists of 95% of all UV radiation emitted by sun and reaching the surface of the earth. UVA exposure usually remains constant throughout the day and the four seasons whereas UVB exposure occurs more during the noon time and in the summer while UVC is almost absorbed completely by O3 never reaching the surface of the earth. UVA penetrates deeper into human skin and causes under-skin damage and under-skin cancers while UVB causes pigmentation, sunburn, photocarcinogenesis. Two kinds of sunscreen agents (UV filters) are currently being used in sunscreens for minimization of these adverse effects: 1. Organic (chemical) filters, e.g. avobenzone, oxybenzone (benzophenone-3, bp-3) or octocrylene, etc.; 2. Inorganic (physical) filters, zinc oxide (ZnO) and titanium dioxide (TiO2). Organic filters absorb UV rays while inorganic filters scatter/reflect UV rays to prevent UV rays’ exposure. In order to cover the whole UV radiation band, UVA and UVB, sunscreens are usually comprised of more than one of these UV filters: organic, in-organic or a combination of both types, which gives broad-spectrum of protection. Beyond its debatable efficiency, questions regarding the main ingredients of different sunscreens are being raised in recent years, mainly about the prevalence of these ingredients about their potential toxicity towards human health.

Since the first day of sunscreen usage in 1930s, the exposure to sunscreen agents has begun. Initially the scope and frequency of the sunscreen usage among the human population was very limited. Therefore, the levels of UV filters found in human samples were usually low. However, for the last 20 years, the situation changed dramatically. In one epidemiological study conducted in 2003-2004, 2517 urine samples from United States (US) general population were analyzed for the presence of oxybenzone (benzophenone-3, bp-3), as part of the 2003-2004 National Health and Nutrition Examination Survey [A.M. Calafat, L.Y.Wong, X.YE, J.A.Reidy, L.L.Needham, Concentrations of the sunscreen agent benzophenone-3 in residents of the United States: National Health and nutrition Examination Survey 2003-2004, Environ. Health Perspect, 116(2008)893-897]. Oxybenzone was detected in 97% of the all samples with mean concentration of 22.9 ng/ml and 95th percentile concentration of 1040 ng/ml. As the data indicated the sunscreen agent occurrences is widespread. Later on another study was carried out focusing on investigating correlation between couples’ presence of urinary benzophenone-type UV filters and sex ratio of their offspring, the mean concentrations of these UV absorber compounds ranged from 0.05 ng/ml to 8.65 ng/ml, with bp-3 as the most predominant among the study population(samples collected between 2005 and 2009 in Michigan and Texas) [J.Bae, S.Kim., K.Kannan, G.M.Buck Louis, Couples’ urinary concentrations of benzophenone-type ultraviolet filters and the secondary sex ratio, Sci. Total Environ. 543(2016)28-36]. In 2007-2009 a study conducted in California among female subjects revealed very surprising findings. About nine times higher than previously reported levels of oxybenzone(up to 13000 ng/ml, average around 200 ng/ml) were found in urine samples collected in 2007-2009 from Californian females, which is probably a result of specific demographics [C.Philippat, D.Bennett, A.M.Calafat, I.H.Picciotto, Exposure to select phthalates and phenols through use of personal care products among Californian Adults and their Children, Environ. Res. 140(2015)369-376]. Compared to the 2003-2004 study the average oxybenzone concentration in urine sample increased more than 9 folds, from 22.9 ng/mL to 200 ng/mL. It was an alarming development.

The experimental studies confirmed substantial absorption and distribution of organic filters whereas inorganic filters seem to penetrate the human skin in a minimal degree. When adults applied a sunscreen formulation containing 10% of oxybenzone, 4-methylbenzylidene camphor (4-MBC) and octyl methoxycinnamate (OMC)on a daily basis (2 mg/cm²) for a week, the mean urine concentrations for these ingredients were 60, 5, 5 ng/ml for females and 140,7, 8 ng/ml for males, respectively [19N.R.Janjua, B.Mogensen, A.M.Andersson, J.H.Petersen, M.Henriksen, N.E.Skakeback, H.C. Wulf; Systemic Absorption of the Sunscreens Benzophenone-3, Octyl-Methoxycinnamate, and 3-(4-Methyl-Benzylidene) Camphor After Whole-Body Topical Application and Reproductive Hormone Levels in Humans, J. Invest.Dermatol.,123(2004)57-61]. At the same time, maximum plasma concentrations for these ingredients, reached 3-4 h after application, were 200, 20, 10 ng/ml for females and 300, 20, 2 ng/ml for males, respectively. Similar findings were reported following a 4-day exposure to these ingredients, which were detectable in the plasma of human males and females merely 2 h following application [20N.R.Janjua, B.Kongshoj, A.M.Andersson, H.C.Wulf, Sunscreens in human plasma and urine after repeated whole-body topical application, J. Eur. Dermatol. Venererol. 22(2008)456-461]. More data on human skin penetration and distribution of various UV filters, both organic and inorganic, can be found in recent reviews.[21 J.Rodriguez, H.I.Maibach, Percutaneous penetration and pharmacodynamics: Wash-in and wash-off of sunscreen and insect repellent, J.Dermatolog. Treatment, 27(2016)11-18; 22,B.Gulson, M.J.Mccall, D.M.Bowman, T.Pinheiro, A review of criticalfactors for assessing the dermal absorption of metal oxide nanoparticles from sunscreens applied to humans, and a research strategy to address current deficiencies Arch. Toxicol. 89(2015)1909-1930; 15, H.Gonzctlez, Percutaneous absorption with emphasis on sunscreens, Photochem.Photobiol.Sci.,9(2010)482-488].

Of importance, some UV filters were also found in human milk samples. In a cohort study between 2004 and 2006, 54 human milk samples were analyzed; UV filters were detectable in 46 samples and levels were positively correlated with the reported usage of UV filter products[23 M.Schlumpf, K.Kypke, M.Wittassek, J.Angerer, H.Mascher, D.Mascher, C. Vökt, M.Birchler, W.Lichtensteiger, Exposure patterns of UV filters, fragrances, parabens, phthalates, organochlor pesticides, PBDEs, and PCBs in human milk: Correlation of UV filters with use of cosmetics, Chemosphere, 8(2010)1171-1183].

In other study, levels of bp-3 in maternal urinary samples taken in gestational weeks 6-30 were positively correlated with the overall weight and head circumference of the baby [24 C.Philippat, M.Mortamais, C.Chevrier, C.Petit, A.M.Calafat, YEX, M.J.Silva, C.Brambilla, I.Pin, M.A.Charles, S.Cordier, R.Slama, Exposure to phthalates and phenols during pregnancy and offspring size at birth, Environ. Health Perspect, 120(2012)464-470]. These reports raise concerns about potential prenatal exposure and developmental toxicity of UV filters.

Although all data has shown exposure to sunscreen agents through usage of sunscreen products is widespread, and it is a very well-known fact, very little research has been done to attempt to improve the bio-safety of sunscreen products by reducing or completely eliminating such exposure via the state of the art formulation techniques.

Our prior PCT Patent Application PCT/CN2018/111881 has summarized the development of sunscreen products and provided a comprehensive review on the widespread biohazards of sunscreen agents caused to human being and the environment. PCT/CN2018/111881 reported an independent research carried out to minimize the biohazards caused by sunscreen agents by reducing the skin penetration of the sunscreen agents via formulation technology. This formulation technology could reduce skin penetration of sunscreen agents by 5 times in average, 9 times at best by a particular formulation.

On May 09, 2019, CNN reported that the Center for Drug Evaluation and Research, an arm of the US Food and Drug Administration, launched a pilot study related to the biosafety of the current sunscreen products on the market. The findings of the study were quite alarming and triggered a government safety investigation. The study found that it took just one day of use for several common sunscreen ingredients to enter the bloodstream at levels high enough to worry the FDA. A study published on May 6, 2019 in the medical journal JAMA also reported that the blood concentration of three of the ingredients continued to rise as daily use continued and then remained in the body for at least 24 hours after sunscreen use ended.

### SUMMARY OF THE INVENTION

The first aspect of the present application is a sunscreen formulation comprising:
(1) a compound A conjugated with an excipient B via a covalent bond; and
(2) a carrier, preferably aqueous carrier, for topical administration.

The covalent bond is an ester or amide bond, preferably an amide bond. The compound A is a sunscreen compound bearing or derivatized with at least one hydroxyl, carboxyl, or amine group. The excipient B is selected from the group consisting of polymers and oligomers, preferably water-soluble polymers and oligomers. Each of the polymers and oligomers preferably has a molecular weight of 500-50000, more preferably 1000-5000. The excipient B bears or is derivatized with a carboxyl functional group when the compound A bears or is derivatized with a hydroxyl or amine group. The excipient B bears or is derivatized with a hydroxyl or amine functional group when the compound A bears or is derivatized with a carboxyl group. The formulation is free of an effective amount of the compound A in a free form. One excipient molecule may be bonded with multiple sunscreen molecules (of the same type or different types that block different UV bands) via multiple ester or amide covalent bonds.

The formulation may be heterogeneous or homogeneous, preferably homogeneous transparent, more preferably an ointment or a viscus solution. The formulation may be aqueous or nonaqueous, preferably aqueous. In some cases, when the formulation needs to be waterproof, then non-aqueous formulation may be advantageous.

The sunscreen compound that bears or is to be derivatized with at least one hydroxyl, carboxyl, or amine group may be selected from the group consisting of avobenzone, cinoxate, dioxybenzone, homosalate, menthyl anthranilate, octocrylene, octyl methoxycinnamate, octyl salicylate, oxybenzone, padimate O, sulisobenzone, trolamine salicylate, and combinations thereof.

The excipient B may be selected from the group consisting of poly acrylic acid, polyvinyl alcohol, polyethylene imine, polyallyl amine, and polyvinyl amine, and combinations thereof, preferably poly acrylic acid and polyethylene imine.

The sunscreen formulation comprises less than 20%, preferably 10%, more preferably less than 5%, particularly preferably less than 1% molar equivalent of the compound A in free form relative to the molar amount of the compound A covalently conjugated with the excipient B.

The sunscreen compound to be derivatized with the carboxyl group may be selected from the group consisting of avobenzone, cinoxate, dioxybenzone, homosalate, menthyl anthranilate, octocrylene, octyl methoxycinnamate, octyl salicylate, oxybenzone, padimate O, sulisobenzone trolamine salicylate, and combinations thereof, and the derivatized sunscreen compound has the following structure of formula (I):

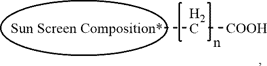

n is 0-18, preferably 4-14-[3-(4-tert-butyl-phenyl)-3-oxo-propionyl]-phenoxy }-butyric acid of the following formula (II):

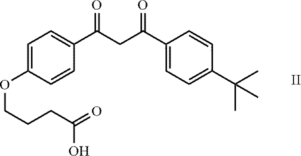 [SMILES: CC(C)(C)c1ccc(C(=O)CC(=O)c2ccc(OCCCC(=O)O)cc2)cc1]

or 4-{4-[3-(4-tert-Butyl-phenyl)-3-oxo-propionyl]-phenoxy}-butyric acid of the following formula (III):

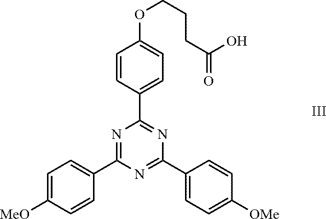 [SMILES: COc1ccc(-c2nc(-c3ccc(OC)cc3)nc(-c3ccc(OCCCC(=O)O)cc3)n2)cc1]

The sunscreen compound to be derivatized with the amine group may be selected from the group consisting of avobenzone, cinoxate, dioxybenzone, homosalate, menthyl anthranilate, octocrylene, octyl methoxycinnamate, octyl salicylate, oxybenzone, padimate O, sulisobenzone trolamine salicylate, and combinations thereof, and the derivatized sunscreen compound may have the following structure of formula (IV):

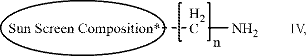

n=0-18, preferably 1-[4-(3-amino-propoxy)-2-hydroxy-phenyl]-3-(4-tert-butyl-phenyl)-propane-1,3-dione of the following formula (V):

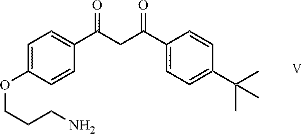 [SMILES: CC(C)(C)c1ccc(C(=O)CC(=O)c2ccc(OCCCN)cc2)cc1]

or (4-aminomethoxy-2-hydroxy-phenyl) phenyl-methanone of the following formula (VI):

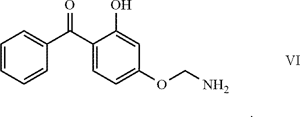

The excipient B may be a polyacrylic acid that is partially esterified, preferably with at least one of C1-C6 lower alcohols, more preferably methanol and/or propanol, with less than 95%, preferably 5%-80%, more preferably 20%-50% of molar amount of carboxyl acid groups of the polyacrylic acid being esterified.

The covalent bond may be the ester bond, and the compound A covalently conjugated with the excipient B is

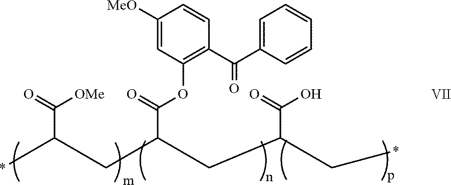

The excipient B may be a random copolymer of three monomers, each of m, n, and p in the formula VII is respectively the molar number of the corresponding monomer unit, with m/(m+n+p) being preferably 20%-50%, n/(m+n+p) being preferably 2%-30%, more preferably 5%-20%, p/(m+n+p) being preferably 40%-70%, or

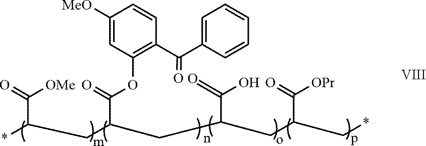

where the excipient B is a random copolymer of four monomers, each of m, n, o, and p in the formula VIII is respectively the molar number of the corresponding monomer unit, with m/(m+n+o+p) being preferably 10%-25%, n/(m+n+o+p) being preferably 2%-30%, most preferably 5%-20%, o/(m+n+o+p) being preferably 40-70%, p/(m+n+o+p) being preferably 10-25%.

The covalent bond may be the amide bond, and the compound A covalently conjugated with the excipient B may be selected from at least one of the following compounds:

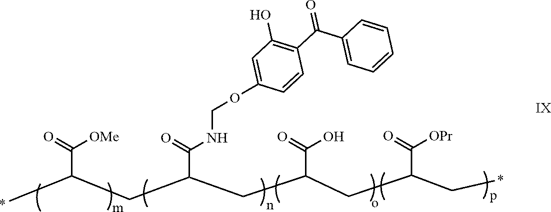

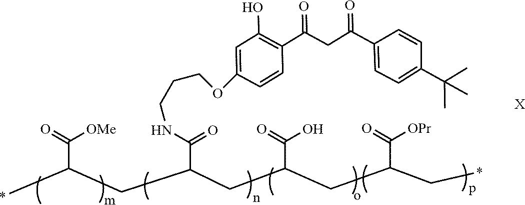

where the excipient B is a random copolymer of four monomers, each of m, n, o, and p is the molar number of the corresponding monomer unit, with m/(m+n+o+p) being preferably 10%-25%, n/(m+n+o+p) being preferably 2%-30%, most preferably 5%-20%, o/(m+n+o+p) being preferably 40%-70%,and p/(m+n+o+p) being preferably 10%-25%,

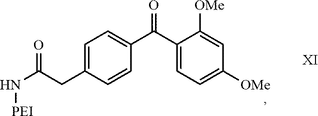

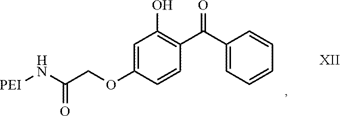

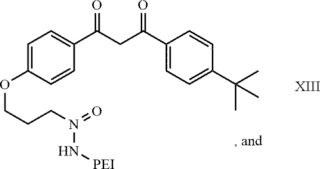

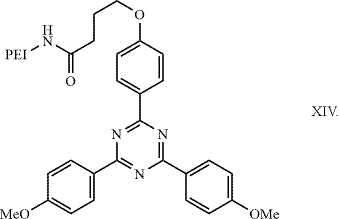

The second aspect of the present application is a method for protecting skin from ultraviolet radiation comprising applying the formulation as discussed above to the skin in need thereof.

The third aspect of the present application is a method of making the above-discussed formulation comprising: reacting the compound A with no greater than (i.e., equal to or less than), preferably 5%-60% (w/w), more preferably 10%-50% (w/w), most preferably 15%-40%, (w/w) based on the total weight of the compound A and the excipient B at a temperature range from ambient temperature (e.g., 20-25° C.) to 80° C. for 5 to 12 hours to obtain the compound A covalently conjugated with the excipient B; and formulating the compound A covalently conjugated with the excipient B with the carrier to obtain the formulation. Preferably the method comprises a step of mixing the compound A covalently conjugated with the excipient B with an amount of the excipient B polymer that is not covalently bonded with the compound A. For example, the amount of the excipient B polymer that is not covalently bonded with the compound A may be 5% to 50% (w/w), preferably 5% to 20% (w/w) relative to the weight the material of the excipient B covalently conjugated with compound A. In the alternative, one may bond a sunscreen agent to the corresponding monomers first, and then co-polymerize the monomers bonded with the sunscreen agent with the underivatized monomer to yield the compound A covalently bonded with the excipient B.

The fourth aspect of the present application is a sunscreen compound with a covalent amide or ester bond having one of the following formulae:

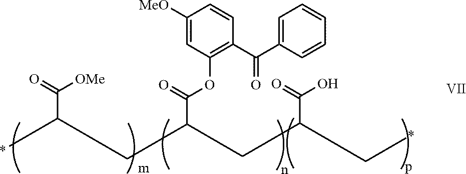

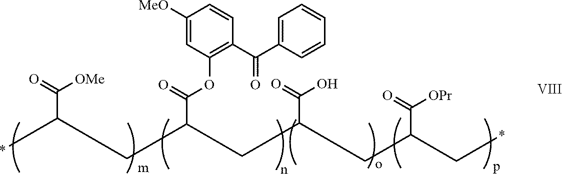

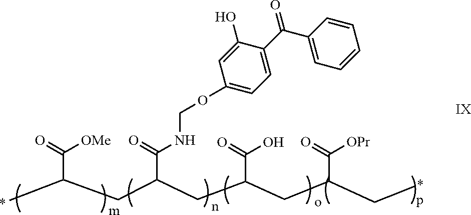

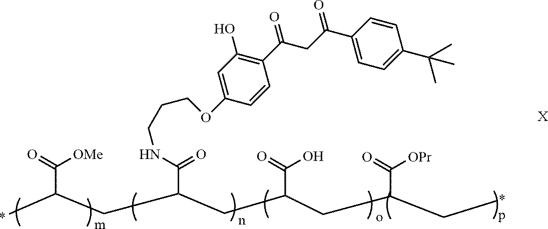

where the polymer unit in each of the formulae is a random copolymer unit of three or four monomers, each of m, n, o, and p is the molar number of the corresponding monomer unit, m/(m+n+o+p) is preferably 10%-25%, n/(m+n+o+p) is preferably 2%-30%, most preferably 5%-20%, o/(m+n+o+p) is preferably 40%-70%, p/(m+n+o+p) is preferably 10%-25%

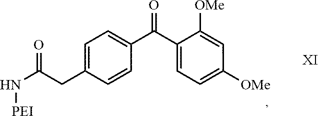

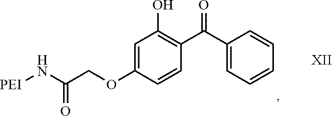

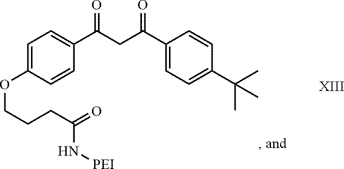

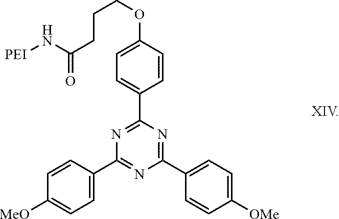

where each of the polymers and oligomers in the above formulas preferably has a molecular weight of 500-50000, more preferably 1000-5000.

### BRIEF DESCRIPTION OF THE DRAWING

FIG. 1 shows oxybenzone-PAA-OMe(50) mice skin penetration test results of Example 22.

FIG. 2 shows octandioic oxybenzone ester formulation mice skin penetration test results of Example 23.

FIG. 3 shows [4-(2,4-dimethoxy-benzoyl)-phenyl]-acetic acid-PEI formulation mice skin penetration test results of Example 24.

FIG. 4 shows the HPLC chromatogram of [4-(2,4-Dimethoxy-benzoyl)-phenyl]-acetic acid Std (0.5 mg/mL) of Example 24.

FIG. 5 shows the HPLC chromatogram of [4-(2,4-Dimethoxy-benzoyl)-phenyl]-acetic acid-PEI formulation test sample of Example 24.

FIG. 6 shows the HPLC chromatogram of (4-Aminomethoxy-2-hydroxy-phenyl)-phenyl-methanone API Std (0.5 mg/mL) of Example 25. NW1701-163.

FIG. 7 shows the HPLC chromatogram of plasma blank of Example 25. NW1701 -163.

FIG. 8 shows the HPLC chromatogram of (4-Aminomethoxy-2-hydroxy-phenyl)-phenyl-methanone-PAA-OMe-OPr(40) formulation test sample (10 mg/mL) of Example 25. NW1701-163

FIG. 9 shows the HPLC chromatogram of (4-Aminomethoxy-2-hydroxy-phenyl)-phenyl-methanone-PAA-OMe-OPr(40) formulation test sample (20 mg/mL) + plasma of Example 25. NW1701-163

FIG. 10 shows the HPLC chromatogram of (4-Aminomethoxy-2-hydroxy-phenyl)-phenyl-methanone-PAA-OMe-OPr(40) formulation plasma sample @5 min of Example 25. NW1701-163

FIG. 11 shows the HPLC chromatogram of (4-Aminomethoxy-2-hydroxy-phenyl)-phenyl-methanone-PAA-OMe-OPr(40) formulation plasma sample @15 min of Example 25. NW 1701-163

FIG. 12 shows the HPLC chromatogram of (4-Aminomethoxy-2-hydroxy-phenyl)-phenyl-methanone-PAA-OMe-OPr(40) formulation plasma sample @30 min of Example 25. NW1701-163

FIG. 13 shows the HPLC chromatogram of (4-Aminomethoxy-2--hydroxy-phenyl)-phenyl-methanone-PAA-OMe-OPr(40) formulation plasma sample @60 min of Example 25. NW1701-163

FIG. 14 shows the HPLC chromatogram of (4-Aminomethoxy-2-hydroxy-phenyl)-phenyl-methanone-PAA-OMe-OPr(40) formulation plasma sample @90 min of Example 25. NW1701-163

FIG. 15 shows the HPLC chromatogram of (4--Aminomethoxy-2-hydroxy-phenyl)-phenyl-methanone-PAA-OMe-OPr(40) formulation plasma sample @120 min of Example 25. NW1701-163

FIG. 16 shows the HPLC chromatogram of plasma blank of Example 26. NW1701-164

FIG. 17 shows the HPLC chromatogram of 1-[4-(3-Amino-propoxy)-2-hydroxyphenyl]-3-(4-tert-butyl-phenyl)-propane-1,3-dione API Std (0.5 mg/mL) of Example 26. NW1701-164

FIG. 18 shows the HPLC chromatogram of 1-[4-(3-Amino-propoxy)-2-hydroxyphenyl]-3-(4-tert-butyl-phenyl)-propane-1,3-dione API Std (0.5 mg/mL) + plasma of Example 26. NW1701-164

FIG. 19 shows the HPLC chromatogram of 1-[4-(3-Amino-propoxy)-2-hydroxyphenyl]-3-(4-tert-butyl-phenyl)-propane-1,3-dione-PAA-OMe-OPr(40) formulation test sample (10 mg/mL) of Example 26. NW1701-164

FIG. 20 shows the HPLC chromatogram of 1-[4-(3-Amino-propoxy)-2-hydroxyphenyl]-3-(4-tert-butyl-phenyl)-propane-1,3-dione-PAA-OMe-OPr(40) formulation test sample (10 mg/mL) + plasma of Example 26. NW 1701-164

FIG. 21 shows the HPLC chromatogram of 1-[4-(3-Amino-propoxy)-2-hydroxyphenyl]-3-(4-tert-butyl-phenyl)-propane-1,3-dione-PAA-OMe-OPr(40) formulation plasma sample @5 min. of Example 26. NW1701-164

FIG. 22 shows the HPLC chromatogram of 1-[4-(3-Amino-propoxy)-2-hydroxyphenyl]-3-(4-tert-butyl-phenyl)-propane-1,3-dione-PAA-OMe-OPr(40) formulation plasma sample @15 min. of Example 26. NW1701-164

FIG. 23 shows the HPLC chromatogram of 1-[4-(3-Amino-propoxy)-2-hydroxyphenyl] -3-(4-tert-butyl-phenyl)-propane-1,3-dione-PAA- OMe-OPr(40) formulation plasma sample @30 min. of Example 26. NW1701-164

FIG. 24 shows the HPLC chromatogram of 1-[4-(3-Amino-propoxy)-2-hydroxyphenyl]-3-(4-tert-btutyl-phenyl)-propane-1,3-dione-PAA-OMe-OPr(40) formulation plasma sample @60 min. of Example 26. NW1701-164

FIG. 25 shows the HPLC chromatogram of 1-[4-(3-Amino-propoxy)-2-hydroxyphenyl] -3-(4-tert-butyl-phenyl)-propane-1,3-dione-PAA- OMe-OPr(40) formulation plasma sample @90 min. of Example 26. NW1701-164

FIG. 26 shows the HPLC chromatogram of 1-[4-(3-Amino-propoxy)-2-hydroxyphenyl]-3-(4-tert-butyl-phenyl)-propane-1,3-dione-PAA-OMe-OPr(40) formulation plasma sample @120 min. of Example 26. NW1701-164

FIG. 27 shows the HPLC chromatogram of plasma blank of Example 27. NW1701-147

FIG. 28 shows the HPLC chromatogram of (4-Benzoyl-3-hydroxy-phenoxy)-acetic acid API Std (0.5 mg/mL) of Example 27. NW1701-147

FIG. 29 shows the HPLC chromatogram of (4-Benzoyl-3-hydroxy-phenoxy)-acetic-acid-PEI formulation test sample (10 mg/mL) of Example 27. NW1701-147

FIG. 30 shows the HPLC chromatogram of (4-Benzoyl-3-hydroxy-phenoxy)-acetic-acid-PEI formulation test sample (10 mg/mL) + plasma of Example 27. NW1701-147

FIG. 31 shows the HPLC chromatogram of (4-Benzoyl-3-hydroxy-phenoxy)-acetic-acid-PEI formulation plasma sample @5 min of Example 27. NW1701-147

FIG. 32 shows the HPLC chromatogram of (4-Benzoyl-3-hydroxy-phenoxy)-acetic-acid-PEI formulation plasma sample @15 min of Example 27. NW1701-147

FIG. 33 shows the HPLC chromatogram of (4-Benzoyl-3-hydroxy-phenoxy)-acetic-acid-PEI formulation plasma sample @30 min of Example 27. NW1701-147

FIG. 34 shows the HPLC chromatogram of (4-Benzoyl-3-hydroxy-phenoxy)-acetic-acid-PEI formulation plasma sample @60 min of Example 27. NW1701-147

FIG. 35 shows the HPLC chromatogram of (4-Benzoyl-3-hydroxy-phenoxy)-acetic-acid-PEl formulation plasma sample @90 min of Example 27. NW1701-147

FIG. 36 shows the HPLC chromatogram of (4-Benzoyl-3-hydroxy-phenoxy)-acetic-acid-PEI formulation plasma sample @120 min of Example 27. NW1701-147

FIG. 37 shows the HPLC chromatogram of plasma blank of Example 28. NW1701-161

FIG. 38 shows the HPLC chromatogram of {4-[3-(4-tert-Butyl-phenyl)-3-oxo-propionyl]-3-hydroxy-phenoxy}-butyric acid API Std (0.5 mg/mL) of Example 28. NW1701-161

FIG. 39 shows the HPLC chromatogram of {4-[3-(4-tert-Butyl-phenyl)-3-oxo-propionyl]-3-hydroxy-phenoxy}-butyric acid API Std (0.5 mg/mL) + plasma of Example 28. NW1701-161

FIG. 40 shows the HPLC chromatogram of {4-[3-(4-tert-Butyl-phenyl)-3-oxo-propionyl]-3-hydroxy-phenoxy}-butyric acid-PEI formulation test sample (lOmg/mL) of Example 28. NW1701-161

FIG. 41 shows the HPLC chromatogram of {4-[3-(4-tert-Butyl-phenyl)-3-oxo-propionyl]-3-hydroxy-phenoxy}-butyric acid-PEI formulation test sample (20 mg/mL) + plasma of Example 28. NW1701-161

FIG. 42 shows the HPLC chromatogram of {4-[3-(4-tert-Butyl-phenyl)-3-oxo-propionyl]-3-hydroxy-phenoxy}-butyric acid-PEI formulation plasma sample @5 min of Example 28. NW1701-161

FIG. 43 shows the HPLC chromatogram of {4-[3-(4-tert-Butyl-phenyl)-3-oxo-propionyl]-3-hydroxy-phenoxy}-butyric acid-PEI formulation plasma sample @15 min of Example 28. NW1701-161

FIG. 44 shows the HPLC chromatogram of {4-[3-(4-tert-Butyl-phenyl)-3-oxo-propionyl]-3-hydroxy-phenoxy}-butyric acid-PEI formulation plasma sample @30 min of Example 28. NW1701-161

FIG. 45 shows the HPLC chromatogram of {4-[3-(4-tert-Butyl-phenyl)-3-oxo-propionyl]-3-hydroxy-phenoxy}-butyric acid-PEI formulation plasma sample @60 min of Example 28. NW1701-161

FIG. 46 shows the HPLC chromatogram of {4-[3-(4-tert-Butyl-phenyl)-3-oxo-propionyl]-3-hydroxy-phenoxy}-butyric acid-PEI formulation plasma sample @90 min of Example 28. NW1701-161

FIG. 47 shows the HPLC chromatogram of {4-[3-(4-tert-Butyl-phenyl)-3-oxo-propionyl]-3-hydroxy-phenoxy}-butyric acid-PEI formulation plasma sample @120 min of Example 28. NW1701-161

### DETAILED DESCRIPTION OF PREFERRED EMBODIMENTS OF THE INVENTION

The following embodiments are provided to illustrate, but not to limit the instant invention.

New organic sunscreen agents with specifically designed functions for formulation purposes were synthesized.

The new sunscreen agents or existing sunscreen agents with specific function groups were reacted with cosmetic excipients, particularly oligomers, polymers and organic molecules, to form chemical covalent bonds between the sunscreen agents and excipient molecules.

The sunscreen agents with —OH groups were reacted with cosmetic excipients, particularly oligomers, polymers bearing carboxy groups and organic molecules bearing carboxy groups, to form covalent ester bonds between the sunscreen agents and excipient molecules.

The sunscreen agents with carboxy groups were reacted with cosmetic excipients, particularly oligomers, polymers bearing —OH groups and organic molecules bearing -OH groups, to form covalent ester bonds between the sunscreen agents and excipient molecules.

The sunscreen agents with COOH groups were reacted with cosmetic excipients, particularly oligomers and polymers bearing primary/secondary amine groups and organic molecules bearing primary/secondary amine groups to form covalent amide bonds between the sunscreen agents and excipient molecules.

The sunscreen agents with amine groups were reacted with cosmetic excipients, particularly oligomers and polymers bearing COOH groups and organic molecules bearing COOH groups, to form covalent amide bonds between the sunscreen agents and excipient molecules.

A formulation scheme of using the materials prepared with excipients as simple organic compounds, oligomers, or polymers covalently bonded with sunscreen agents was developed. The covalent bonding includes ester or amide bonds were designed aiming at reducing/eliminating skin penetration of sunscreen agents.

The present invention relates to a preparation of sunscreen materials and a novel formulation technique to reduce the skin penetration of sunscreen agents to improve biosafety of sunscreen products or completely stop the skin penetration of sunscreen agents to render the sunscreen product completely biosafe.

The present inventors have made intensive research to develop new active sunscreen agents bearing specifically designed function groups, to prepare sunscreen formulation materials of excipients bonded covalently with sunscreen agents, and a formulation scheme for these sunscreen formulation materials which will reduce skin penetration of sunscreen agents or at best eliminate the skin penetration of sunscreen agents for the ultimate purposes of enhancing the biosafety of sunscreen products.

Accordingly, it is an objective of this invention to provide a synthesis of sunscreen agents which bears a carboxyl function group, —COOH, specifically designed for the purpose of preparing the sunscreen formulation materials.

Accordingly, it is an objective of this invention to provide a synthesis of sunscreen agents which bears an amine function group, —NH₂, specifically designed for the purpose of preparing sunscreen formulation materials.

It is another objective of this invention to provide a synthesis of a sunscreen formulation material of excipients containing carboxy function groups covalently bonded with sunscreen agents which bears a hydroxy function group, —OH, via ester bonds.

It is another objective of this invention to provide a synthesis of a sunscreen formulation material of excipients containing —OH function groups covalently bonded with sunscreen agents which bears a carboxy function group, —COOH, via ester bonds.

In one aspect of the present invention there is provided a synthesis of a sunscreen formulation material of excipients containing carboxy function groups covalently bonded with sunscreen agents which bears an amine function group, —NH₂, via amide bonds to reduce skin penetration of sunscreen agents or stop the skin penetration of sunscreen agents for the ultimate purposes of enhancing the bio-safety of sunscreen products.

In another aspect of the present invention, there is provided a synthesis of a sunscreen formulation material of excipients containing primary/secondary amine function groups, —NH₂/—NH, covalently bonded with sunscreen agents which bears a carboxy function group, —COOH, via amide bonds to reduce skin penetration of sunscreen agents or stop the skin penetration of sunscreen agents for the ultimate purposes of enhancing the bio-safety of sunscreen products.

In still another aspect of the present invention, there is provided a formulation scheme using sunscreen formulation materials of excipients covalently bonded with sunscreen agents via ester or amide bonds to reduce skin penetration of sunscreen agents or stop the skin penetration of sunscreen agents for the ultimate purposes of enhancing the bio-safety of sunscreen products.

### EXAMPLES

The invention is illustrated herein by the experiments described by the following examples, which should not be considered as limiting. Those skilled in the art will understand that this invention may be embodied in many different forms and should not be constructed as limited to the embodiments set forth herein. Rather, these embodiments are provided so that this disclosure will fully convey the invention to those skilled in the art. Many modifications and other embodiments of the invention will come to mind in one skilled in the art to which this invention pertains having the benefit of the teachings presented in the foregoing description. Although specific terms are employed, they are used as in the art unless otherwise indicated.

### Example 1

### Synthesis of 4-{4-[3-(4-Tert-Butyl-Phenyl)-3-Oxo-Propionyl]-Phenoxy}-Butyric Acid

Synthesis scheme:

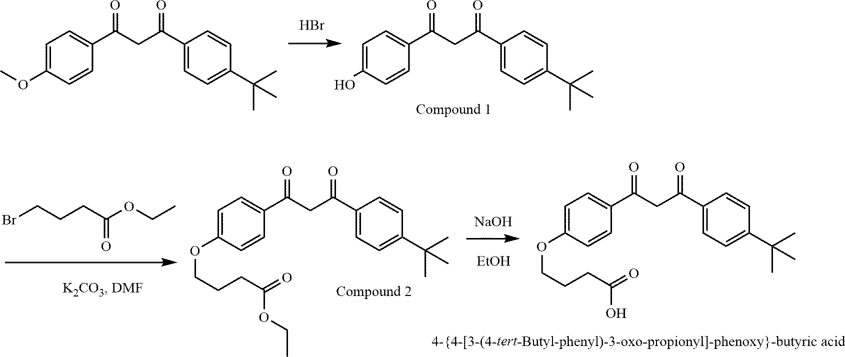

Synthesis of intermediate compound 1

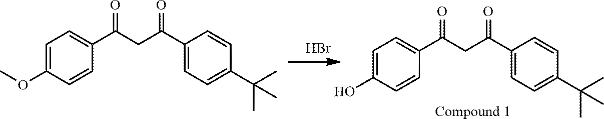

Dissolve avobenzone (5.0 g) in acetic acid (50 mL). To the solution add HBr 48% solution (20 mL). Under N₂ stir and reflux the mixture for 16 hours. After concentrating the reaction solution, add saturated brine (100 mL) to quench the reaction. Extract the product with ethyl acetate (EtAc) (30 mL × 3). Dry ethyl acetate extract solution with Na₂SO₄. Filter out the Na₂SO₄ and concentrate the solution. The column purification with hexane:ethyl acetate(5:1) as eluent yields compound 1 (13.0 g), a colorless solid. The yield is 62.9%

Synthesis of intermediate compound 2

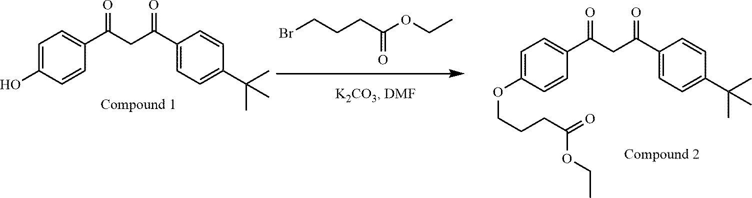

Dissolve compound 1 (2.0 g) and 4-bromo-butyric acid ethyl ester (1.34 g) in DMF (15 mL). Add K₂CO₃ (1.4 g) and stir the mixture at 60° C. for 5 hours under N₂. Add water (50 mL) to quench the reaction. Filter and collect the solid. Purify the solid with column with eluent hexane:ethyl acetate(10:1). Compound 2, a colorless solid (2.4 g), was obtained in 87% yield.

Synthesis of 4-{4-[3-(4-tert-Butyl-phenyl)-3-oxo-propionyl]-3-hydroxy-phenoxy}-butyric acid

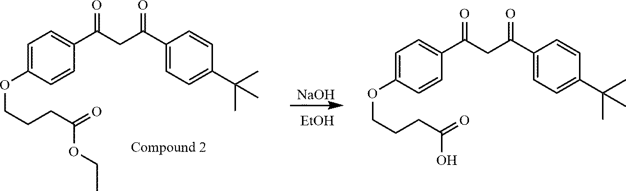

Dissolve compound 2 (2.0 g) in ethanol (20 mL). Add NaOH (0.39 g) and water (12 mL). Stir the mixture at ambient temperature for 12 hours. Concentrate the reaction mixture by removing organic volatile solvent. Add 2N HCl to adjust pH to 4-5. A lot of solid was produced. Filter and wash the solid with water. After drying, light yellow solid product (1.8 g) was obtained, in 97% yield.

### Example 2

### Synthesis of 1-[4-(3-Amino-Propoxy)-2-Hydroxy-Phenyl]-3-(4-Tert-Butyl-Phenyl)-Propane-1,3-Dione

Synthesis scheme:

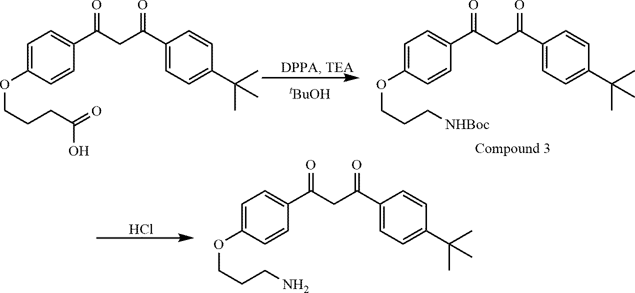

1-[4-(3-Amino-propoxy)phenyl]-3-(4-tert-butyl-phenyl)-propane-1,3-dione

Synthesis of intermediate compound 3

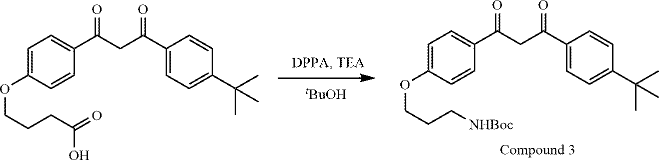

Dissolve 4-{4-[3-(4-tert-Butyl-phenyl)-3-oxo-propionyl]-phenoxy}-butyric acid (1.0 g) in ^{t}BuOH (10 mL). Add TEA (0.53 g) and DPPA (1.4 g). Stir the mixture and reflux for 5 hours. Concentrate the reaction mixture and purify via a column chromatography with hexane:ethyl acetate (5:1). Light yellow oily material (1.1 g) was obtained in 93% yield.

Synthesis of 1-[4-(3-Amino-propoxy)-2-hydroxy-phenyl]-3-(4-tert-butyl-phenyl)-propane-1,3-dione

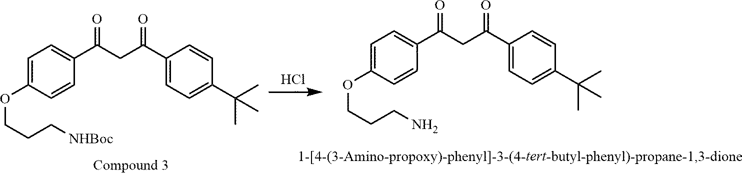

Dissolve compound 3 (1.0 g) in dichloromethane (10 mL). To the solution add trifluoroacetic acid (5 mL). Stir the mixture at ambient temperature for 12 hours. After concentrating the reaction solution, add saturated Na₂CO₃, to adjust the pH to about 8. Extract the product with ethyl acetate and dry ethyl acetate extract solution with Na₂SO₄. Filter out the Na₂SO₄, concentrate the filtrate. Column purification yields product (0.6 g), yellowish solid. The yield is 77%.

### Example 3

### Synthesis of 4-{4-[4,6-Bis-(4-Methoxy-Phenyl)-[1,3,5]Triazin-2-Yl]-Phenoxy}-Butyric Acid

Synthesis scheme:

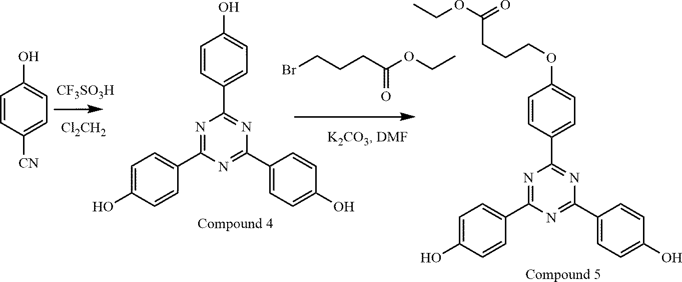

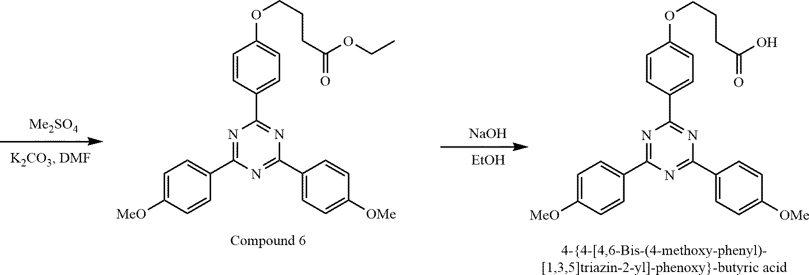

Synthesis of intermediate compound 4

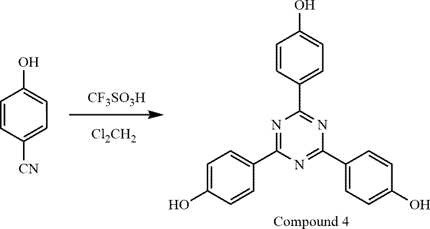

Dissolve 4-Hydroxy-benzonitrile (2.0 g) in dichloromethane (20 mL). Cool it below 0° C. and add CF₃SO₃H (7.5 g) dropwise under N₂. After completing the addition of CF₃SO₃H, warm up to ambient temperature and stir the mixture for 12 hours. Remove solvent, then add ice water (50 mL). A lot of colorless solid was yielded. Adjust the pH with NH₄OH within 4-5 and filter out the solid. Wash the solid with plenty of water. After drying, colorless solid (1.92 g), intermediate compound 4, was obtained in 96% yield.

Synthesis of intermediate compound 5

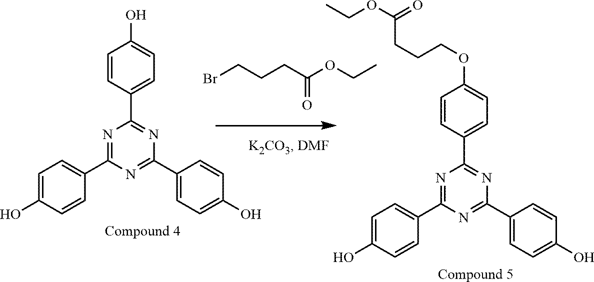

Dissolve compound 4, 4-bromo-butyric acid ethyl ester (0.864 g), and K₂CO₃ in DMF (20 mL). Stir the mixture at 40° C. for 8 hours. Pour the reaction mixture to water (50 mL). Extract the product with plenty of ethyl acetate. Ethyl acetate solution was dried over Na₂SO₄. Filter and concentrate the extract solution. Column purification yielded yellow solid, intermediate compound 5, in 48% yield.

Synthesis of intermediate compound 6

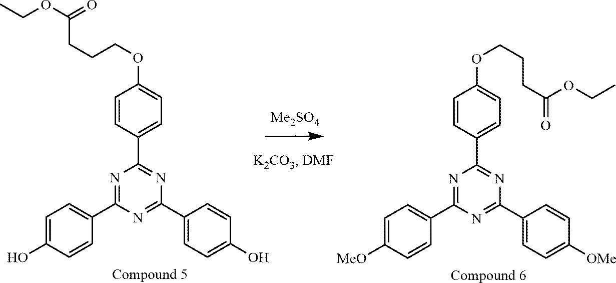

Dissolve compound 5 (1.0 g), K₂CO₃ (1.2 g) in acetone (10 mL). Add Me₂SO₄ (1.07 g) at ambient temperature while stirring. Once completing the addition, reflux the reaction mixture at 60° C. and stir for 8 hours. Pour the reaction mixture to water (50 mL). Extract the product with ethyl acetate (30 mL X 2). Dried over Na₂SO₄, filter and concentrate the filtrate. Column purification yields compound 6 (0.8 g), light yellow solid, in 75% yield.

Synthesis of 4-{4-[4,6-Bis-(4-methoxy-phenyl)-[1,3,5]triazin-2-yl]-phenoxy}-butyric acid

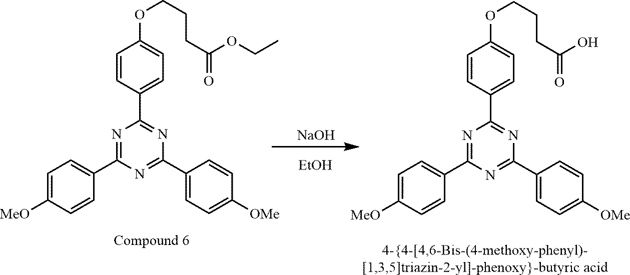

Dissolve compound 6 (0.72 g) in ethanol (10 mL). Add NaOH (0.115 g) and water (7 mL). Stir the mixture at ambient temperature for 8 hours. Concentrate the reaction mixture by removing organic volatile solvent. Add 3N HCl to adjust pH to 4-5. A lot of solid was produced. Filter and wash the solid with water. After drying, light yellow solid product (0.680 g) was obtained, with a yield of 100%.

### Example 4

### Preparation of Oxybenzone-PAA-OMe(50) Material

Note:(Oxybenzone-PAA-OMe(50) = 50 represents —COOH groups of PAA are capped randomly with —OMe in 50%)

Synthesis of polyacrylic acid (PAA) modified with 50% methyl ester [PAA-OMe(50)]: Take PAA solid 4.42 g (—COOH moles 4.42/72=61.30 mmole), add MeOH ~80 mL and dissolve completely. At 0° C., add SOCl₂ 2.25 mL (0.0613x50%x118.97/1.63=2.24 mL) and 4 drops of DMF. Stir at 0° C. for 30 min. Warm up to ambient temperature and stir for 12 hours. Remove excessive MeOH under vacuum to yield 5.35 g solid. Extract PAA-OMe(50) with ethyl acetate 50 mL. Separate the precipitate (0.36 g), remove some solvent and a thick PAA-OMe(50) viscus solution was obtained.

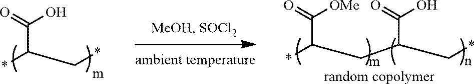

Preparation of PAA-OMe(50)-COCl: Take an aliquot of PAA-OMe(5) EtAc solution and remove all volatile solvent. A thick viscus material (2.03 g) was obtained (—COOH moles:2.03/72x0.50=14.1 mmoles). Add CH₂Cl₂ (10 mL) to dissolve PAA-OMe(50). Add SOCl₂ (4.89 g, 41.10 mmoles, 3 mL) to the PAA-OMe(50) solution in CH₂Cl₂ at ambient temperature and add 3 drops of DMF. Stir for half an hour at ambient temperature, then raise the temperature to ~80° C. for 9 hours. Remove solvent and excessive SOCl₂ under vacuum at 80° C. to yield a thick viscus material.

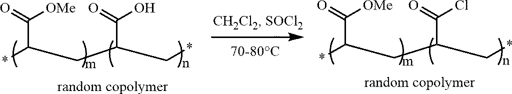

At ambient temperature add oxybenzone (0.65 g, 2.85 mmole, 0.65/228.47). Stir the mixture and add NEt₃ slowly to neutralize HCl produced to maintain the pH of the reaction mixture at 8-9. Stir for 12 hours at ambient temperature. Crude oxybenzone-PAA-OMe(50) material was obtained.

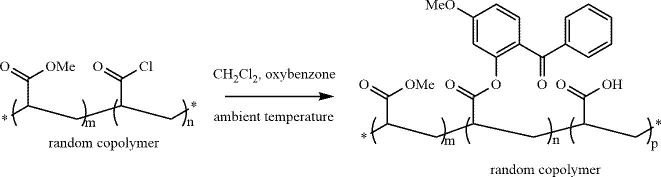

Purification of Oxybenzone-PAA-OMe(50): Load the crude Oxybenzone-PAA-OMe(50) onto a column, elute with plenty of Hex:EtAc(5:1) until there is no free oxybenzone. Use CH₂Cl₂:MeOH(5:1) to elute the product Oxybenzone-PAA-OMe(50).

### Example 5

### Preparation of Oxybenzone-PAA-OMe-OPr(30) Material

Preparation of PAA—OMe—OPr(30): Take PAA solid (1.72 g) (—COOH moles 1.72/72=23.90 mmole), add MeOH ~10 mL to dissolve PAA. Add n-propanol 10 mL and mix thoroughly. At ambient temperature, add SOCl₂ (0.52 mL) (0.0239x30%x118.97/1.63=0.52 mL). The solution turned clear. Add 3 drops of DMF and stir the mixture for 5 hours. Remove all solvents under vacuum with a mechanic pump. PAA—OMe—OPr(30), a viscus solid, was obtained.

Preparation of PAA—OMe—OPr(30)-COCl: Add CH₂Cl₂ 20 mL to dissolve the viscus solid of PAA—OMe—OPr(30%). Add SOCl₂ (2.0 mL) at ambient temperature and add 3 drops of DMF. Stir for an hour at ambient temperature, then raise the temperature to 50-55° C. Stir the mixture at 50-55° C. for 3 hours. Remove solvent and unconsumed SOCl₂ under vacuum with mechanic pump. PAA—OMe—OPr(30)—COCl, a thick viscus material, was obtained.

Add 10 mL of CH₂Cl₂ to dissolve the PAA—OMe—OPr(30)—COCl. At ambient temperature add oxybenzone (1.02 g). Stir the mixture and add NEt₃ slowly to neutralize HCl produced to maintain the pH of the reaction mixture at 8-9. Stir for 5 hours, then remove solvent. Wash the solids with ethyl acetate several times until no free oxybenzone is detected with TLC. The solids were extracted with CH₂Cl₂, filtered. After removing the solvent a light beige colored solid (1.91 g) was obtained.

### Example 6

Preparation of Octandioic Oxybenzone Ester.

Add octandioic acid (0.39 g) to CH₂Cl₂ (10 mL). Add SOCl₂ (1 mL) and 3 drops of DMF. Stir the mixture until all solids are dissolved. Remove solvent and excessive SOCl₂ under vacuum. Add anhydrous CH₂Cl₂ (10) mL and oxybenzone (0.78 g). Stir the mixture for 8 hours. Quench the reaction with saturated saline. Add saturated NaHCO₃ aqueous solution to adjust pH and extract with ethyl acetate. The crude product was purified with silico-gel column and eluent Hex:EtAc(10:1) until there is no free oxybenzone. The di-substituted and mono-substituted products are eluted with Hex:EtAc(5:1). Removing eluent, a mixture of di-substituted and mono-substituted oxybenzone esters are obtained.

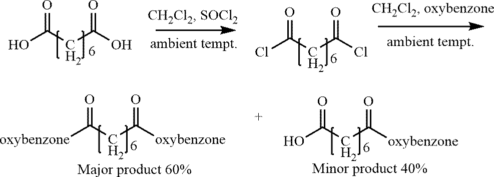

### Example 7

### Preparation of [4-(2,4-Dimethoxy-Benzoyl)-Phenyl]-Acetic Acid-Polyethylene Imine Material

Take [4-(2,4-Dimethoxy-benzoyl)-phenyl]-acetic acid(0.50 g), HBTU (0.76 g) to dissolve in DMSO (10 mL). Add DIPEA (0.32 g) and stir for 30 min. at ambient temperature. Add polyethylene imine (PEI) (2.01 g) and stir the reaction for 12 hours. Quench the reaction with saturated saline (30 mL) and extract with EtAc. Drying over Na₂SO₄. Removing EtAc under vacuum yields [4-(2,4-Dimethoxy-benzoyl)-phenyl]-acetic acid-PEI material (2.34 g). TLC confirmed that there is no free [4-(2,4-Dimethoxy-benzoyl)-phenyl]-acetic acid.

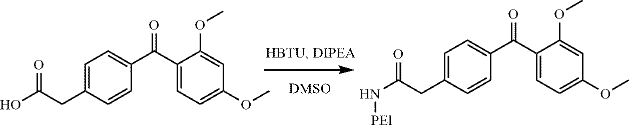

### Example 8

### Preparation of (4-Aminomethoxy-2-Hydroxy-Phenyl)-Phenyl-Methanone-PAA-OMe-OPr(40) Material

Preparation of PAA—OMe—OPr(40): Add MeOH (25 mL) to dissolve PAA solid (5.52 g) (—COOH moles = 5.52/72=76.7 mmole). Add n-propanol 20 mL and mix thoroughly. At 0.0° C., add SOCl₂ (2.24 mL) (0.0767x40%x118.97/1.63 = 2.24 mL). Add 3 drops of DMF and warm up to ambient temperature. Stir the mixture for 3 hours at ambient temperature. Remove all solvents under vacuum with a mechanic pump. A viscus solid of PAA—OMe—OPr(40) was obtained.

Preparation of PAA—OMe—OPr(40)—COCl: Add CH₂Cl₂ (15 mL) to dissolve the viscus solid of PAA—OMe—OPr(40) (1.67 g). Add SOCl₂ (2.5 mL) at 0° C. and 3 drops of DMF. Stir the reaction for an hour at ambient temperature. Raise the temperature to 50-55° C. and stir the mixture for 5 hours. Remove solvent and unconsumed SOCl₂ under vacuum with mechanic pump. A viscus product of PAA—OMe—OPr(40)COCl was obtained.

Add 12.5 mL of CH₂Cl₂ to dissolve the PAA—OMe—OPr(40)—COCl prepared as above. At 0° C. add the solution of (4-Aminomethoxy-2-hydroxy-phenyl)-phenyl-methanone (0.40 g) in 5 mL CH₂Cl₂. Add NEt₃ slowly to neutralize HCl produced to maintain the pH of the reaction mixture at 8-9. Stir the reaction for 1 hour at 0° C., then stir the reaction for 12 hours at ambient temperature. Orange precipitate was formed. Remove CH₂Cl₂ and add H₂O (10 mL) and adjust pH to 2-3 with 2N HCl. Add EtAc (20 mL) to extract product. EtAc layer was washed with H₂O (20 mL). Removing EtAc yielded a light beige solid (1.44 g).

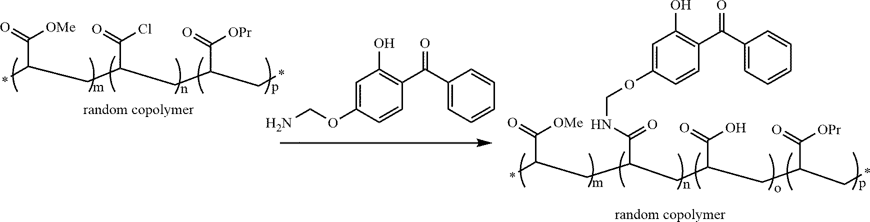

### Example 9

### Preparation of 1-[4-(3-Amino-Propoxy)-2-Hydroxy-phEnyl]-3-(4-Tert-Butyl-Phenyl)-Propane-1,3-Dione-PAA-OMe-OPr(40) Material

Preparation of PAA—OMe—OPr(40): Follow the procedures described in the section of Preparation of PAA—OMe—OPr(40) of example 8.

Preparation of PAA—OMe—OPr(40)—COCl: Add CH₂Cl₂ (15 mL) to dissolve the viscus solid of PAA—OMe—OPr(40) (1.62 g). Add SOCl₂ (2.5 mL) at 0° C. and 3 drops of DMF. Warm up to ambient temperature and stir the reaction for an hour. Raise the temperature to 50° C. and stir the mixture for 5 hours. Remove solvent and unconsumed SOCl₂ under vacuum with mechanic pump. A viscus product of PAA—OMe—OPr(40)—COCl was obtained.

Add CH₂Cl₂ (12 mL) to dissolve the PAA—OMe—OPr(40)—COCl prepared as above. At 0° C. add the solution of 1-[4-(3-Amino-propoxy)-phenyl]-3-(4-tert-butyl-phenyl)-propane-1,3-dione (0.400 g) in 5 mL CH₂Cl₂ and NEt₃ slowly to neutralize HCl produced to maintain the pH of the reaction mixture at 8-9. Stir the reaction for 1 hour at 0° C., then stir the reaction for 12 hours at ambient temperature. Orange precipitate was formed. Remove CH₂Cl₂. Add H₂O (10 mL) and adjust pH to 2-3 with 2N HCl. Add EtAc (20 mL) to extract product. EtAc layer was washed with H₂O (20 mL). Removing EtAc yielded a light beige solid (1.87 g).

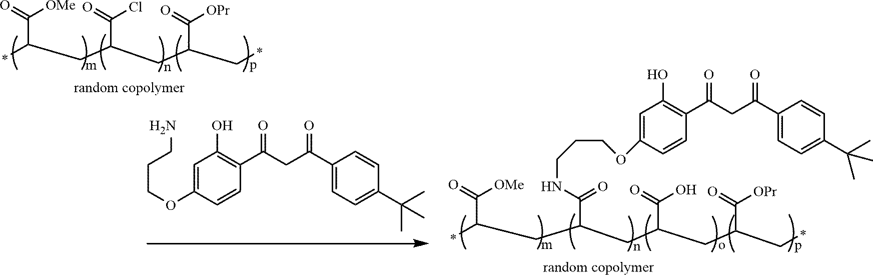

### Example 10

### Preparation of (4-Benzoyl-3-Hydroxy-Phenoxy)-Acetic Acid-PEI Material

Take (4-Benzoyl-3-hydroxy-phenoxy)-acetic acid (0.50 g), HBTU (0.84 g) to dissolve in DMF (10 mL). Add DIPEA (0.356 g) in DMF (5 mL) and stir for 30 min. at ambient temperature. Dissolve PEI (0.79 g) in a mixture DMF (10 mL) and DMSO (3 mL). Add (4-Benzoyl-3-hydroxy-phenoxy)-acetic acid/HBTU/DIPEA solution to the PEI solution. The reaction was stirred for 12 hours. Quench the reaction with 2N HCl (20 mL). Wash with CH₂Cl₂/MeOH (10:1) 15 mLX2. Wash with CH₂Cl₂/MeOH (5:1) 10 mLX2 until no (4-Benzoyl-3-hydroxy-phenoxy)-acetic acid was detected with TLC. Removing H₂O with mechanic vacuum pump yields (4-Benzoyl-3-hydroxy-phenoxy)-acetic acid-PEl material (4.52 g), a rubbery material.

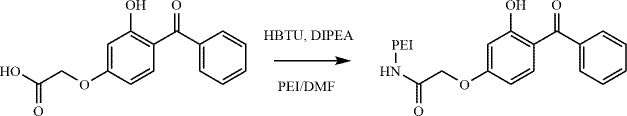

Dialysis of (4-Benzoyl-3-hydroxy-phenoxy)-acetic acid-PEI material: Weigh NaOH (42 mg) to dissolve in H₂O (10 mL). Add water to (4-Benzoyl-3-hydroxy-phenoxy)-acetic acid-PEI material. Add 3 mL of NaOH solution to the (4-Benzoyl-3-hydroxy-phenoxy)-acetic acid-PEI water solution. Mix well. Transfer to a dialysis bag (100 cut off) to undergo dialysis. Concentrate(4-Benzoyl-3-hydroxy-phenoxy)-acetic acid-PEI water solution, then add 2^{nd} batch of NaOH solution (3 mL) and continue the dialysis until no Cl⁻ is detected in dialysis water phase. Removing H₂O from (4-Benzoyl-3-hydroxy-phenoxy)-acetic acid-PEI water solution yielded an orange viscus material of (4-Benzoyl-3-hydroxy-phenoxy)-acetic acid-PEI.

### Example 11

### Preparation of 4-{4-[3-(4-Tert-Butyl-Phenyl)-3-Oxo-Propionyl]-3-Hydroxy-Phenoxy}-Butyric Acid-PEI Material

Take 4-{4-[3-(4-tert-Butyl-phenyl)-3-oxo-propionyl]-3-hydroxy-phenoxy}-butyric acid (0.49 g), HBTU (0.60 g) to dissolve in DMF (10 mL). Stir for 30 min. Add the solution of DIPEA (0.25 g) in 2 mL DMF and stir for 30 min. at ambient temperature. Add the above solution to the solution of PEI (0.77 g) in DMF (11 mL) and stir the reaction for 12 hours. Quench the reaction with 2N HCl at 0° C. and adjust pH to 2-3. Add H₂O (30 mL) and a clear solution was obtained. Wash the solution with CH₂Cl₂/MeOH(10:1) 15 mLX2 and CH₂Cl₂/MeOH(5:1) 10 mL.

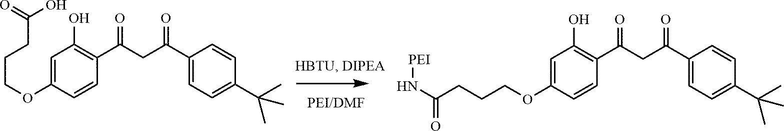

Dialysis of 4-{4-[3-(4-tert-Butyl-phenyl)-3-oxo-propionyl]-3-hydroxy-phenoxy}-butyric acid-PEI material: The solution of 4-{4-[3-(4-tert-Butyl-phenyl)-3-oxo-propionyl]-3-hydroxy-phenoxy}-butyric acid--PEI was subjected to dialysis with dialysis bag (100 cut off). The dialysis was terminated until no Cl⁻ can be detected in the dialysis water. Removing H₂O from 4-{4-[3-(4-tert-Butyl-phenyl)-3-oxo-propionyl]-3-hydroxy-phenoxy}-butyric acid-PEI water solution yielded an orange viscus material.

### Example 12

### Preparation of 4-{4-[4,6-Bis-(4-Methoxy-phenyl)-[1,3,5]Triazin-2-Yl]-Phenoxy}-Butyric Acid-PEI Material

Take 4-{4-[4,6-Bis-(4-methoxy-phenyl)-[1,3,5]triazin-2-yl]-phenoxy}-butyric acid (0.52 g), HBTU (0.51 g) to dissolve in DMF (5 mL). Stir for 15 min. Add the solution of DIPEA (0.22 g) in DMF (2 mL) and stir for 30 min. at ambient temperature. Colorless precipitate was formed. Add the above suspension solution to the solution of PEI (0.80 g) in DMF (8 mL) and stir the reaction for 12 hours. Colorless precipitate disappeared. Quench the reaction with saturated brine at 0° C. and adjust pH to 2-3 with 2N HCl. A clear solution was obtained. Wash the solution with CH₂Cl₂/MeOH(10: 1) 15 mLX2.

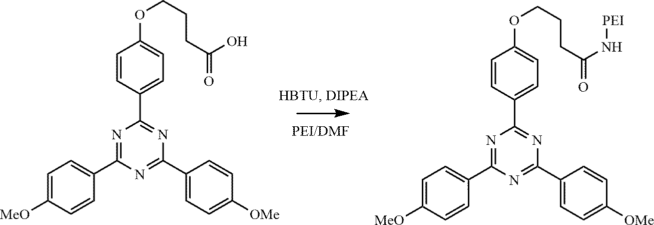

Dialysis of 4-{4-[4,6-Bis-(4-methoxy-phenyl)-[1,3,5]triazin-2-yl]-phenoxy}-butyric acid-PEI material: The solution of 4-{4-[4,6-Bis-(4-methoxy-phenyl)-[1,3,5]triazin-2-yl]-phenoxy}-butyric acid-PEI was subjected to dialysis with dialysis bag (100 cut off). The dialysis was terminated until no Cl⁻ can be detected in the dialysis water. Removing H₂O from 4-{4-[4,6-Bis-(4-methoxy-phenyl)-[1,3,5]triazin-2-yl]-phenoxy}-butyric acid-PEI water solution yielded an orange viscus material.

### Example 13

### Oxybenzone-PAA-OMe(50) Formulation

Oxybenzone-PAA-OMe(50) Formulation Composition

| | | | |
|---|---|---|---|
| Oxybenzone-PAA-OMe(50) | PEG 200 | PEG 400 | PEG 1500 |
| 1.05 g | 0.59 g | 0.33 g | 1.13 g |

Add PEG 200 to Oxybenzone-PAA-OMe(50). Mix thoroughly. Add PEG 400 and mix thoroughly. Add PEG 1500 to the above homogeneous solution. Heat gently to dissolve PEG 1500 until a homogeneous solution was obtained. The % content of oxybenzone was determined by calculation based on UV absorbance data to be 7.4%.

### Oxybenzone/[4-(2,4-Dimethoxy-Benzoyl)-Phenyl]-Acetic Acid Control Formulation

For the purpose of comparison, a oxybenzone/[4-(2,4-Dimethoxy-benzoyl)-phenyl]-acetic acid control formulation was prepared. Lack of the covalent interaction between oxybenzone/[4-(2,4-Dimethoxy-benzoyl)-phenyl]-acetic acid with excipient matrix is the base as a control standard. The composition is listed in the following table.

Control Formulation Composition for Oxybenzone-PAA-OMe(50) Formulation

| | | | | |
|---|---|---|---|---|
| Oxybenzone | [4-(2,4-Dimethoxy-benzoyl)-phenyl]-acetic acid | PEG 200 | PEG 400 | PEG 1500 |
| 148.52 mg | 199.82 mg | 0.02 g | 0.10 g | 0.53 g |

Weigh each component, oxybenzone, [4-(2,4-Dimethoxy-benzoyl)-phenyl]-acetic acid, PEG 200, PEG 400 and PEG 1500, into a small beaker. The mixture was heated in a 35° C. oven and was mixed thoroughly until a clear solution was obtained. Cooling to room temperature yielded an ointment.

### Example 14

### Octandioic Oxybenzone Ester Formulation

Octandioic oxybenzone ester Formulation composition

| | | | |
|---|---|---|---|
| Octandioic oxybenzone ester | PEG 200 | PEG 400 | PEG 1500 |
| 0.84 g (oxybenzone=333.56 mg) | 1.04 g | 0.46 g | 1.49 g |

Add PEG 200 to octandioic oxybenzone ester. Mix thoroughly with gentle heating until all octandioic oxybenzone ester was dissolved. Add PEG 400 and PEG 1500 and mix thoroughly with gentle heating until all PEG 1500 was dissolved. A homogeneous solution was obtained. The % content of oxybenzone was determined by calculation based on UV absorbance data to be 8.71%.

### Oxybenzone/[4-(2,4-Dimethoxy-Benzoyl-Phenyl]-Acetic Acid Control Formulation

Same as the one of example 13

### Example 15

### (2,4-Dimethoxy-Benzoyl)-Phenyl]-Acetic Acid-PEI Formulation

[4-(2,4-Dimethoxy-benzoyl)-phenyl]-acetic acid-PEI Formulation Composition

| | | |
|---|---|---|
| Sunscreen material | PAA(30%) | PEG 200 |
| 1.33 g (oxybenzone=238 mg) | 1.35 g | 0.59 g |

Add MeOH (5 mL) to [4-(2,4-Dimethoxy-benzoyl)-phenyl]-acetic acid-PEI material (1.33 g) to complete dissolve. Add PEG 200 (0.71 g) and mix thoroughly. Evaporate MeOH under vacuum. Add PEG 1500 (0.34 g) and mix thoroughly with mild heating. The % content of oxybenzone was determined by calculation based on UV absorbance data to be 10.0%.

### Oxybenzone/[4-(2,4-Dimethoxy-Benzoyl)-Phenyl]-Acetic Acid Control Formulation

Same as the one of example 13

### Example 16

### Aminomethoxy-2-Hydroxy-Phenyl)-Phenyl-Methanone-PAA-OMe-OPr(40) Formulation

(4-Aminomethoxy-2-hydroxy-phenyl)-phenyl-methanone-PAA-OMe-OPr(40) Formulation Composition

| | | |
|---|---|---|
| Sunscreen material | PAA(30%) | PEG 200 |
| 1.40 g (avobenzone= 187.88 mg) | 0.24 g | 1.24 g |

Add EtAc (2.80 g) and acetic acid (0.43 g) to the (4-Aminomethoxy-2-hydroxyphenyl)-phenyl-methanone—PAA—OMe—OPr (40) material (1.40 g) to mix. Add PEG 200 (0.21 g) and PAA 30% solution (0.24 g) to mix thoroughly. Add PEG 200 (1.03 g) and mix thoroughly. Evaporate volatile solvents in a 37° C. oven. Finally, a mass (3.04 g) of ointment was obtained. The % content of oxybenzone was determined by calculation based on UV absorbance data to be 6.18%.

### Oxybenzone/[4-(2,4-Dimethoxy-Benzoyl)-Phenyl]-Acetic Acid Control Formulation

Same as the one of example 13

### Example 17

### 1-(3-Amino-Propoxy)-2-Hydroxy-Phenyl]-3-(4-Tert-Butyl-Phenyl)-Propane-1,3-Dione—PAA—OMe—OPr (40) Formulation

1-[4-(3-Amino-propoxy)-2-hydroxy-phenyl]-3-(4-tert-butyl-phenyl)-propane-1,3-dione—PAA—OMe—OPr (40) Formulation Composition

| | | |
|---|---|---|
| Sunscreen material | PAA(30%) | PEG 200 |
| 1.81 g (avobenzone=213 mg) | 1.35 g | 0.59 g |

Add ethyl acetate (2.50 g) to the 1-[4-(3-Amino-propoxy)-2-hydroxy-phenyl]-3-(4-tert-butyl-phenyl)-propane-1,3-dione-PAA-OMe-OPr(40) material (1.81 g) to complete dissolve. Add acetic acid (1.10 g) and mix thoroughly. Evaporate EtAc. Add acetic acid (1.09 g) and mix thoroughly. Evaporate ethyl acetate. Add PAA water solution (30%) (0.68 g) and mix well. Add more PAA water solution (30%) 0.67 g and mix well. Evaporate volatile solvents in an 37° C. oven. Add PEG 200 (0.59 g) and mix well. The % content of avobenzone was determined by calculation based on UV absorbance data to be 5.34%.

### Avobenzone Control Formulation

Avobenzone Control Formulation

| | | | |
|---|---|---|---|
| Avobenzone | Iso-Propanol | PEG 200 | PEG 1500 |
| 101.51 mg | 104.92 mg | 1130.46 mg | 742.77 mg |

Weigh PEG 200 and PEG 1500 into a beaker. The beaker was placed in a 35° C. oven. Mix until a clear solution was obtained. Weigh avobenzone into the container containing PEG. Mix thoroughly. A clear solution was obtained.

### Example 18

### Benzoyl-3-Hydroxy-Phenoxy)-Acetic Acid-PEI Formulation

(4-Benzoyl-3-hydroxy-phenoxy)-acetic acid-PEI Formulation Composition

| | | | |
|---|---|---|---|
| Sunscreen material | PEI | PEG 200 | PEG 1500 |
| 0.84 g (oxybenzone=200.4 mg) | 0.46 g | 0.66 g | 0.73 g |

Add PEI 0.46 g to (4-Benzoyl-3-hydroxy-phenoxy)-acetic acid-PEI material (0.84 g) and mix well to complete dissolve. Add PEG 200 (0.66 g) and mix thoroughly. Add PEG 1500 (0.73 g) and mix thoroughly with mild heating. The % content of oxybenzone was determined by calculation based on UV absorbance data to be 7.5%.

### Oxybenzone/[4-(2,4-Dimethoxy-Benzoyl)-Phenyl]-Acetic Acid Control Formulation

Same as the one of example 13.

### Example 19

### 4-{4-(4-Tert-Butyl-Phenyl)-3-Oxo-Propionyl]-3-Hydroxy-Phenoxy}-Butyric Acid-PEI Formulation

4-{4-[3-(4-tert-Butyl-phenyl)-3-oxo-propionyl]-3-hydroxy-phenoxy}-butyric acid-PEI Formulation Composition

| | | |
|---|---|---|
| Sunscreen material | PEI | PEG 200 |
| 2.50 g (avobenzone=206.75 mg) | 0.63 g | 1.16 g |

Add PEI (0.63 g) to 4-[4-[3-(4-tert-Butyl-phenyl)-3-oxo-propionyl]-3-hydroxy-phenoxy]-butyric acid-PEl material (2.50 g) and mix well to complete dissolve. Add PEG 200 (1.16 g) and mix thoroughly. The % content of avobenzone was determined by calculation based on UV absorbance data to be 4.82%.

### Avobenzone Control Formulation

Same as the one of example 17.

### Example 20

### 4-{4-[4,6-Bis-(4-Methoxy-Phenyl)-[1,3,5]Triazin-2-Yl]-Phenoxy}-Butyric Acid-PEI Formulation

4-{4-[4,6-Bis-(4-methoxy-phenyl)-[l,3,5]triazin-2-yl]-phenoxy}-butyric acid--PEI Formulation Composition

| | | |
|---|---|---|
| Sunscreen material | PEI | H₂O |
| 1.64 g (=triazine348.5 mg) | 0.69 g | 0.46 g |

Add PEI (0.69 g) to 4-{4-[4,6-Bis-(4-methoxy-phenyl)-[1,3,5]triazin-2-yl]-phenoxy}-butyric acid-PEI material (1.64 g) and H₂O (1.05 g) mix well to complete dissolve. Evaporate H₂O until proper viscosity was reached. An ointment material (2.79 g) was obtained. The % content of was determined by calculation based on UV absorbance data to be 12.5%.

### Example 21

### Animal Test Results

Mice test procedures:

Mice preparation and blood sample taking: A group of mice ranging from 16 to 60 were fed with adequate food and water for 3 days. Twelve hours before the test the food was removed, and only water was given. Usually 12 mice were randomly selected with 2 mice assigned for each time points of 5, 15, 30, 60, 90, 120 minutes. Sometimes 3 mice may be selected for certain sample time points. Two hours before the test the mice were subjected to anesthesia and the hair on the back (2.5--3.0 cm²) was removed. After recovering from anesthesia mice will be applied with testing ointment sample. The exact same testing procedures will be carried out with the control standard sample. At 5, 15, 30, 60, 90, 120 min. Blood sample (1 mL) will be collected after removing the eyes.

Blood sample pretreatment: after the blood sample was taken, it was immediately subjected to centrifugation 10 min. at 12000 rpm, supernatant plasma layer was taken, preserved at -20° C. for later use.

Blood sample preparation: Accurately transfer 150 µL plasma sample to a 1.5 mL EP test tube. Add methanol and acetonitrile 200 µL, respectively. Vertex mix for 1 min., then centrifuge for 10 min. at 12000 rpm. Precisely transfer the supernatant solution to another test tube. At 40° C. blow N₂ through to dryness. To the solids left add methanol 150 µL, vertex mix for 1 min. centrifuge at 12000 rpm for 10 min. Supernatant solution was used directly for HPLC analysis.

HPLC analysis:

Oxybenzone-PAA-OMe(50): Column: C18 RP column (250 x 4.6 mm, 5 m); column temperature: 25° C.; Mobile phase: MeOH:H₂O=75:25; Flow rate: 1.0 mL/min.; Wavelength: 290 nm; Injection volume: 20 uL NW1701-125

Octandioic oxybenzone ester: same as above.

(2,4-Dimethoxy-benzoyl)-phenyl]-acetic acid-PEI: Column: C18 RP column (250 x 4.6 mm, 5 µm); column temperature: 25° C.; mobile phase: MeOH:0.05% Formic Acid = 60:40; Flow rate: 1.0 mL/min.; Wavelength: 260 nm, 290 nm; Injection volume: 20 L;

Oxybenzone and (2,4-Dimethoxy-benzoyl)-phenyl]-acetic acid Control Sample: same as above. NW1701-128

(4-Aminomethoxy-2-hydroxy-phenyl)-phenyl-methanone-PAA-OMe-OPr(40): Column: C18 RP column (250 × 4.6 mm, 5 µm); column temperature: 25° C.; mobile phase: MeOH:0.05% Formic Acid = 70:30; Flow rate: 1.0 mL/min.; Wavelength: 310 nm; Injection volume: 20 L;

1-[4-(3-Amino-propoxy)-2-hydroxy-phenyl1-3-(4-tert-butyl-phenyl)-propane)-1,3-dione-PAA-OMe-OPr(40): Column: C18 RP column (250 × 4.6 mm, 5 m); column temperature: 25° C.; mobile phase: MeOH:0.05% Formic Acid = 80:20; Flow rate: 1.0 mL/min.; Wavelength: 360 nm; Injection volume: 20 µL;

(4-Benzoyl-3-hydroxy-phenoxy)-acetic acid-PEI: Column: C18 RP column (250 x 4.6 mm, 5 µm); column temperature: 25° C.; mobile phase: MeOH:0.05% Formic Acid = 60:40; Flow rate: 1.0 mL/min,; Wavelength: 260 nm; Injection volume: 20 µL;

{4-[3-(4-tert-Butyl-phenyl)-3-oxo-propionyl]-3-hydroxy-phenoxy}-butyric acid-PEI: Column: C18 RP column (250 × 4.6 mm, 5 µm); column temperature: 25° C.; mobile phase: MeOH:0.05% Formic Acid = 80:20; Flow rate: 1.0 mL/min.; Wavelength: 360 nm; Injection volume: 20 µL;

4-{4-[4,6-Bis-(4-methoxy-phenyl)-[l,3,5]triazin-2-yl]-phenoxy}-butyric acid--PEI: Column: C18 RP column (250 × 4.6 mm, 5 µm); column temperature: 25° C.; mobile phase: MeOH:0.05% Formic Acid = 90:10; Flow rate: 1.0 mL/min.; Wavelength: 310 nm; Injection volume: 20 µL;

### Example 22

### Oxybenzone-PAA-OMe(50) Material Formulation Mice Skin Penetration Test Results

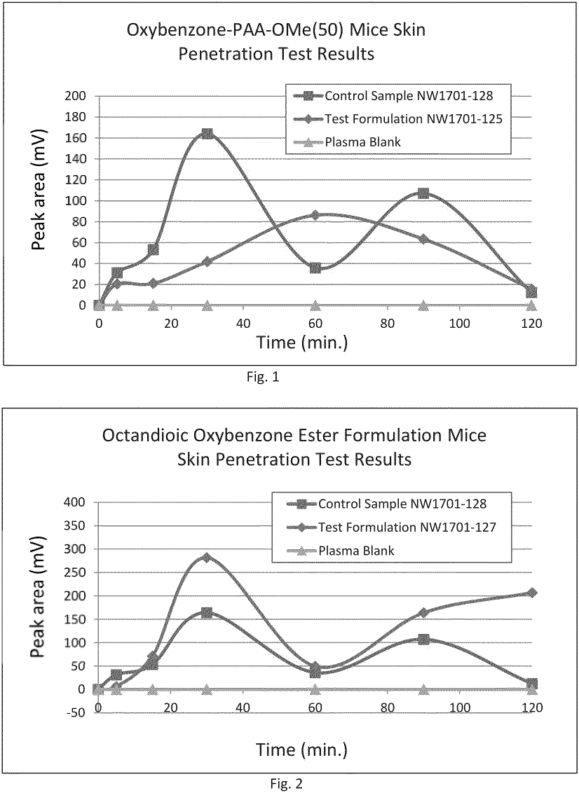

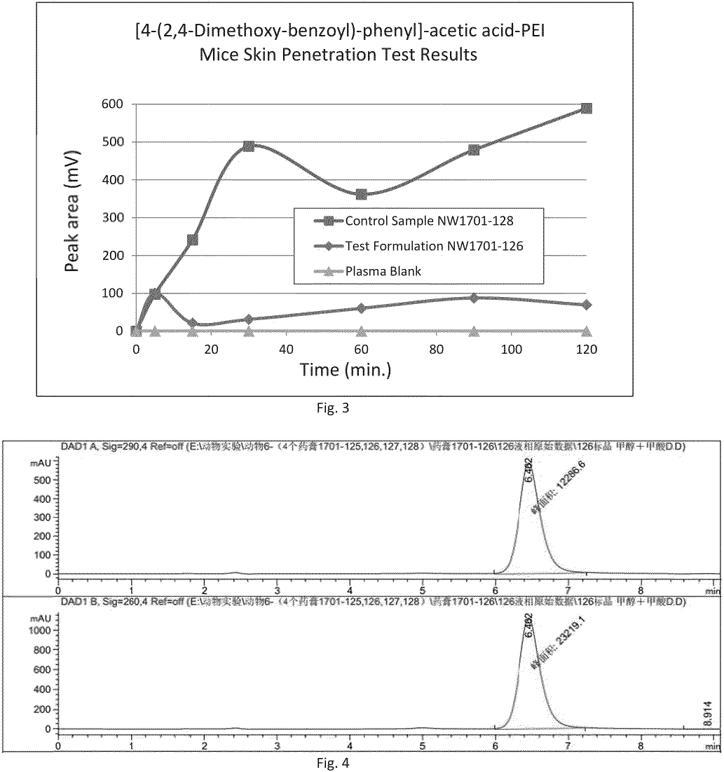

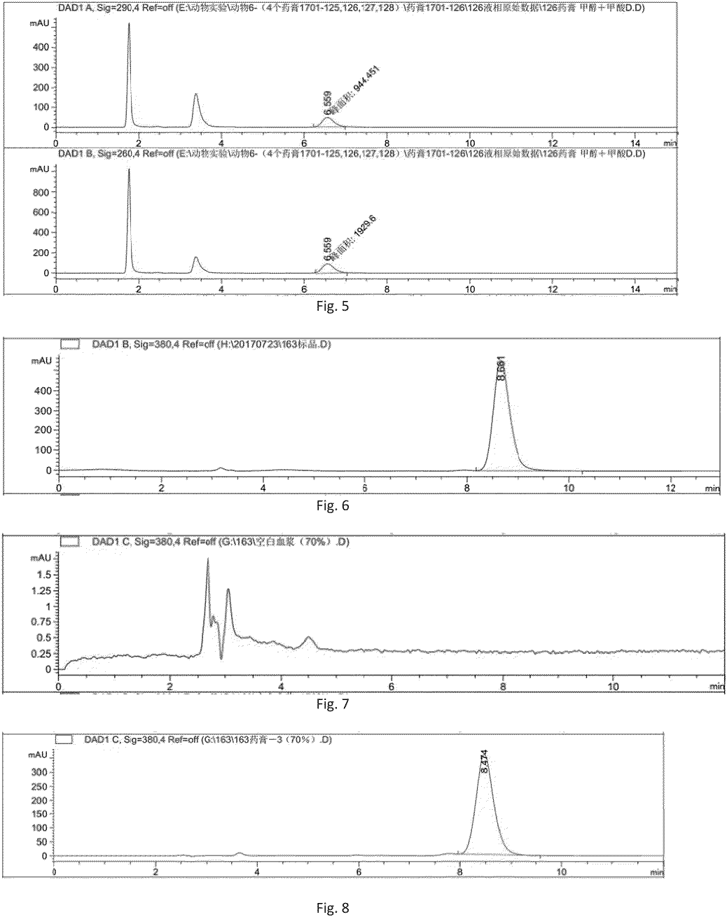

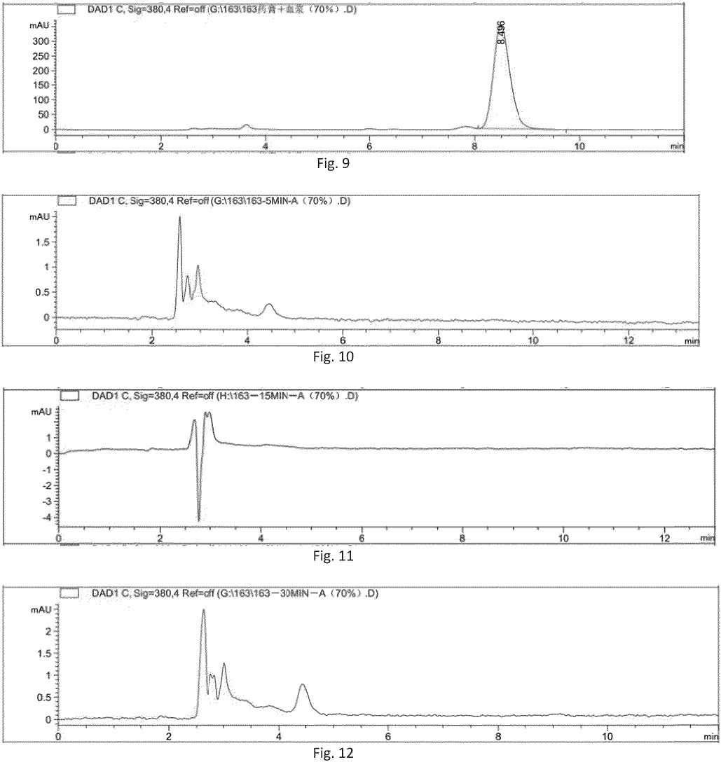

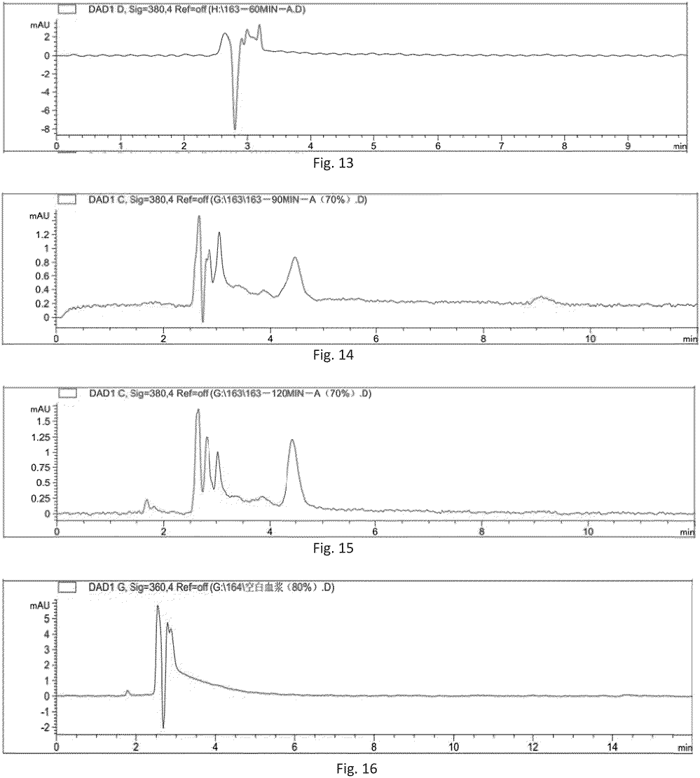

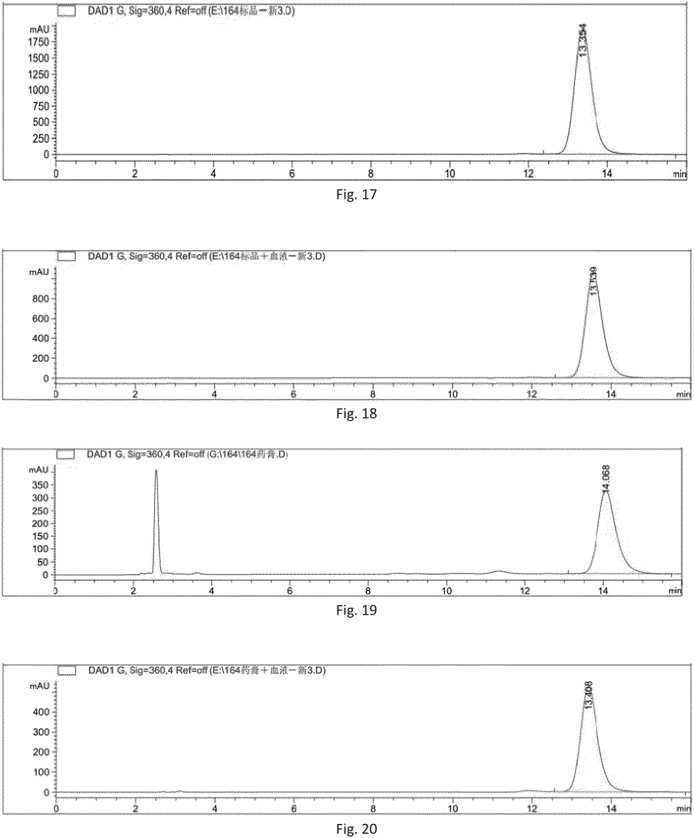

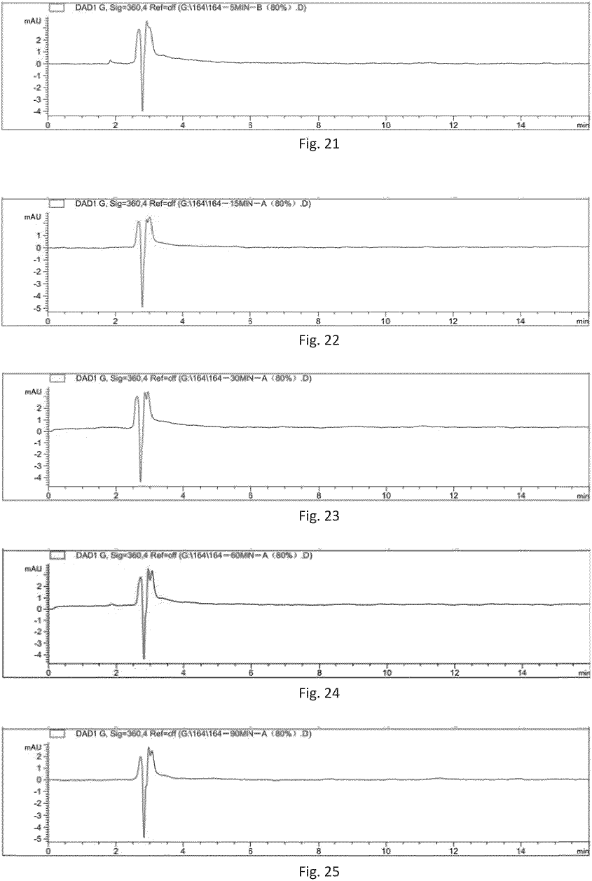

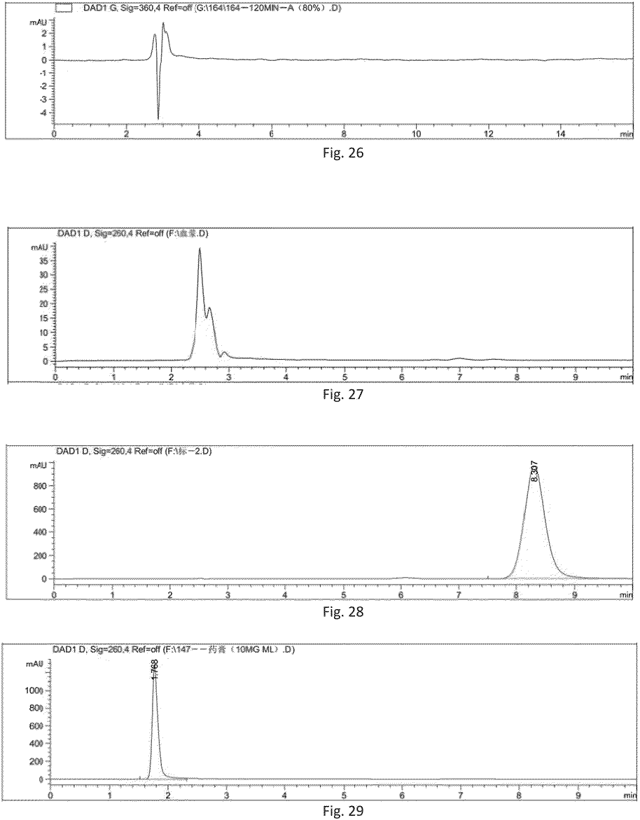

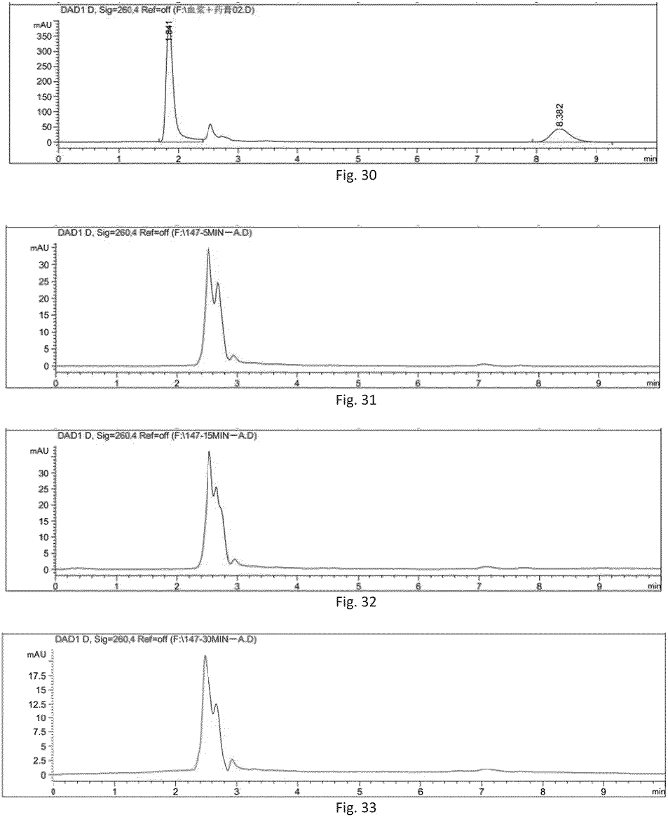

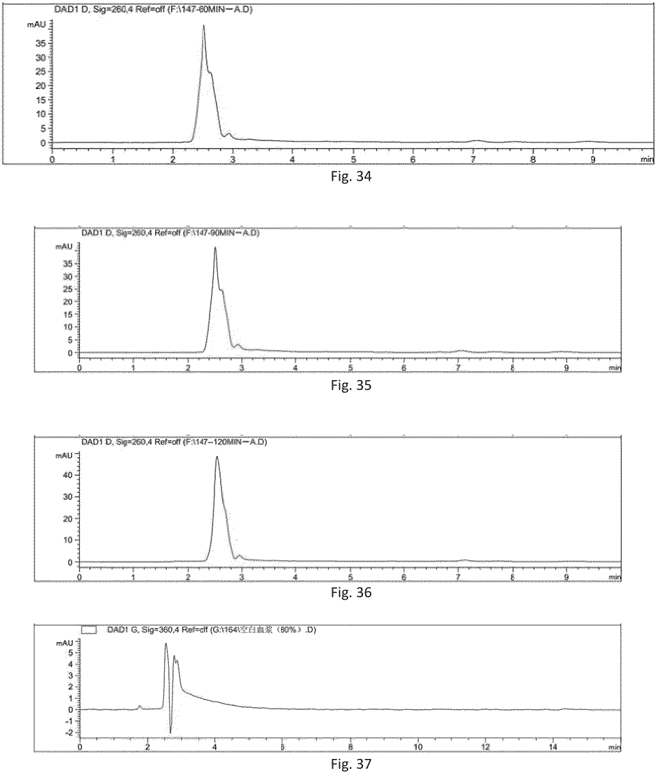

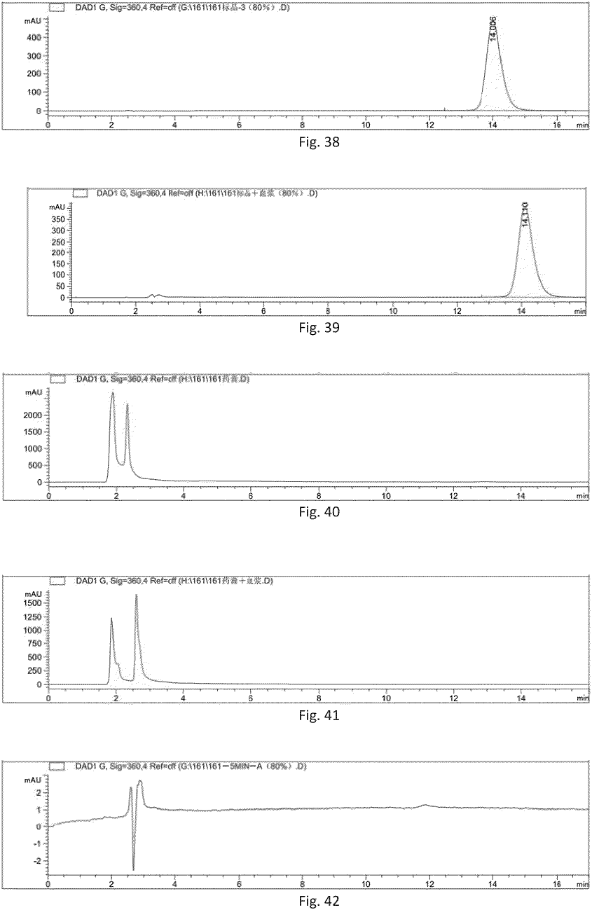

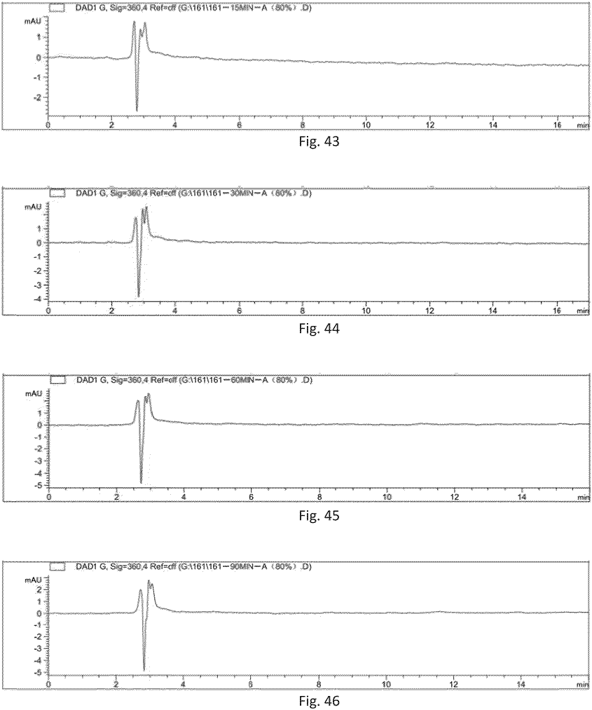

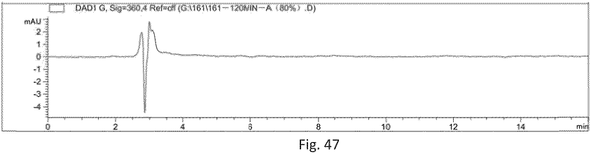

As shown in FIG. 1, the Oxybenzone-PAA-OMe(50) material formulation failed to completely stop the oxybenzone skin penetration, however, did yield a significant reduction in oxybenzone skin penetration. The research in the laboratory demonstrates the hydrolysis of Oxybenzone-PAA-OMe(50) material to yield free oxybenzone which is the cause of the unwanted skin penetration. The concentration of oxybenzone in the blood sample for control formulation are significantly higher than the concentrations of oxybenzone for the test formulation samples. The area under curve (AUC), representing the exposure of oxybenzone, for the control sample is 2.24 times of the AUC of the test formulation.

### Example 23

### Octandioic Oxybenzone Ester Formulation Mice Skin Penetration Test Results

No free oxybenzone was detected in the sunscreen formulation sample with HPLC. After 5 min, significant amount of oxybenzone were detected in the mice blood samples, however, no octandioic oxybenzone esters was detected, indicating skin penetration of octandioic oxybenzone ester and rapid hydrolysis of octandioic oxybenzone ester in the plasma. oxybenzone concentration profile in blood for octandioic oxybenzone ester test sample is quite similar to that for free oxybenzone control sample.

### Example 24

The retention time for [4-(2,4-Dimethoxy-benzoyl)-phenyl]-acetic acid is 6.462 min. The HPLC data of the test sample NW1701 -126 indicates the existence of free [4--(2,4-Dimethoxy-benzoyl)-phenyl]-acetic acid at 6.559 min. in the sample. The contamination is due to unsuccessful purification process. The mice skin penetration test results demonstrate a significant 84% reduction in skin penetration of [4-(2,4-Dimethoxy-benzoyl)-phenyl]-acetic acid. The elimination of contaminating free [4-(2,4-Dimethoxy-benzoyl)-phenyl]-acetic acid could result in zero skin penetration.

### Example 25

The HPLC data indicate that no peaks at 8.661 min. for (4-Aminomethoxy-2-hydroxy-phenyl)-phenyl-methanone was detected in all plasma samples, indicating skin penetration of (4-Aminornethoxy-2-hydroxy-phenyl)-phenyl-methanone was completely eliminated. Zero skin penetration was successful achieved.

### Example 26

The HPLC data indicate the retention time of the sunscreen agent, 1-[4-(3-Amino-propoxy)-2-hydroxy-phenyl]-3-(4-tert-butyl-phenyl)-propane-1,3-dione was 13.35 min. The blending of 164 test sample and plasma did yield a component eluted at 13.408 min, indicating possible hydrolysis of 1-[4-(3-Amino-propoxy)-2-hydroxy-phenyl]-3-(4-tert-butyl-phenyl)-propane-1,3-dione-PAA-OMe-OPr(40). However, no 1-[4-(3-Amino-propoxy)-2-hydroxy-phenyl]-3-(4-tert-butyl-phenyl)-propane-1,3-dione was detected in all plasma samples, indicating skin penetration of 1-[4-(3-Amino-propoxy)-2-hydroxy-pheny1]-3-(4-tert-butyl-phenyl)-propane-1,3-dione was completely eliminated. Zero skin penetration was successful achieved.

### Example 27

The HPLC data indicate the retention time of the sunscreen agent, (4-benzoyl-3-hydroxy-phenoxy)-acetic acid was 8.307 min. The blending of NW1701-147 test sample and plasma did yield a component eluted at 8.382 min, indicating possible hydrolysis of (4-Benzoy1-3--hydroxy--phenoxy)--acetic acid-PEl material. However, no (4-Benzoy1-3-hydroxy-phenoxy)-acetic acid was detected in all plasma samples, indicating skin penetration of (4-Benzoy1-3-hydroxy--phenoxy)--acetic acid was eliminated. Zero skin penetration was successfully achieved.

### Example 28

The HPLC data indicate the retention time of the sunscreen agent, 4-{4-[3-(4-tert-Buty1-pheny1)-3-oxo-propiony1]-3-hydroxy-phenoxy}-butyric acid was 14.007 min. The blending of NW1701-161 test sample and plasma did not yield a component eluted at 14.007 min, indicating no hydrolysis of 4- {4-[3-(4-tert-Butyl-pheny1)-3-oxo-propiony1]-3-hydroxy-phenoxy}-butyric acid-PEl material. No 4-{4-[3-(4-tert-Butyl-phenyl)-3-oxo-propiony1]-3-hydroxy-phenoxy}-butyric acid was detected in all plasma samples, indicating skin penetration of4-{4-[3-(4-tert-Butyl-phenyl)-3-oxo-propionyl]-3-hydroxy-phenoxy}-butyric acid was completely eliminated. Zero skin penetration was successful achieved.

The invention is not limited by the embodiments described above which are presented as examples only but can be modified in various ways within the scope of protection defined by the appended patent claims.

What is claimed is:

1. A method of making a sunscreen formulation, wherein the sunscreen formulation comprises:
(1) a compound A conjugated with an excipient B via a covalent bond; and
(2) a carrier, for topical administration,
wherein the covalent bond is an ester or amide bond;
the compound A is a sunscreen compound bearing or derivatized with at least one hydroxyl, carboxyl, or amine group;
the excipient B is selected from the group consisting of polymers and oligomers
the excipient B bears or is derivatized with a carboxyl functional group when the compound A bears or is derivatized with a hydroxyl or amine group;
the excipient B bears or is derivatized with a hydroxyl or amine functional group when the compound A bears or is derivatized with a carboxyl group; and
the formulation is free of an effective amount of the compound A in a free form,
the method comprises:
reacting the compound A and the excipient B at a temperature range from ambient temperature to 80° C. for 5 to 12 hours to obtain the compound A covalently conjugated with the excipient B; and
formulating the compound A covalently conjugated with the excipient B with the carrier to obtain the formulation.

2. The method of claim 1 wherein the formulation is heterogeneous or homogeneous.

3. The method of claim 1 wherein the sunscreen compound that bears or is to be derivatized with at least one hydroxyl, carboxyl, oramine group is selected from the group consisting of avobenzone, cinoxate, dioxybenzone, homosalate, menthyl anthranilate, octocrylene, octyl methoxycinnamate, octyl salicylate, oxybenzone, padimate O, sulisobenzone, trolamine salicylate and combinations thereof.

4. The method of claim 1 wherein the excipient B is selected from the group consisting of poly acrylic acid, polyvinyl alcohol, polyethylene imine, polyallyl amine, and polyvinyl amine, and combinations thereof, preferably poly acrylic acid and polyethylene imine.

5. The method of claim 1 comprising less than 20% molar equivalent of the compound A in free form relative to the molar amount of the compound A covalently conjugated with the excipient B.

6. The method of claim 1 wherein the sunscreen compound to be derivatized with the carboxyl group is selected from the group consisting of avobenzone, cinoxate, dioxybenzone, homosalate, menthyl anthranilate, octocrylene, octyl methoxycinnamate, octyl salicylate, oxybenzone, padimate O, sulisobenzone trolamine salicylate, and combinations thereof, and the derivatized sunscreen compound has the following structure of formula (I): 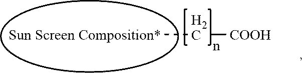
n is 0-18.

7. The method of claim 1 wherein the sunscreen compound to be derivatized with the amine group is selected from the group consisting of avobenzone, cinoxate, dioxybenzone, homosalate, menthyl anthranilate, octocrylene, octyl methoxycinnamate, octyl salicylate, oxybenzone, padimate O, sulisobenzone trolamine salicylate, and combinations thereof, and the derivatized sunscreen compound has the following structure of formula (IV): 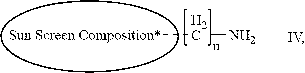
n=0-18.

8. The method of claim 1 wherein the excipient B is a polyacrylic acid that is partially esterified, with less than 95% of molar amount of carboxyl acid groups of the polyacrylic acid being esterified.

9. The method of claim 1 wherein the covalent bond is the ester bond, and the compound A covalently conjugated with the excipient B is 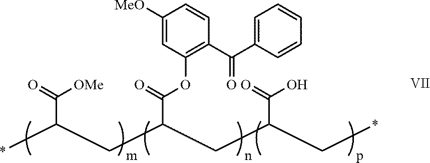
wherein the excipient B is a random copolymer of three monomers, each of m, n, and p in the formula VII is respectively the molar number of the corresponding monomer unit, with m/(m+n+p) being 20%-50%, n/(m+n+p) being 2%-30%, p/(m+n+p) being 40%-70%, or 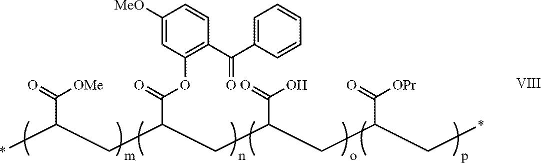
wherein the excipient B is a random copolymer of four monomers, each of m, n, o, and p in the formula VIII is respectively the molar number of the corresponding monomer unit, with m/(m+n+o+p) being 10%-25%, n/(m+n+o+p) being 2%-30%, o/(m+n+o+p) being 40-70%, and p/(m+n+o+p) being 10%-25%.

10. The method of claim 1 wherein the covalent bond is the amide bond, and the compound A covalently conjugated with the excipient B is selected from at least one of the following compounds: 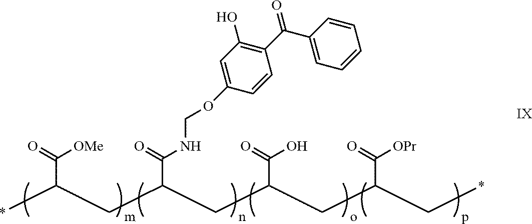 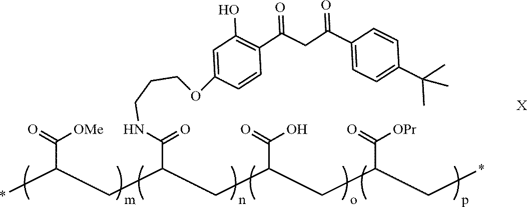
wherein the excipient B is a random copolymer of four monomers, each of m, n, o, and p is the molar number of the corresponding monomer unit, with m/(m+n+o+p) being 10%-25%, n/(m+n+o+p) being 2%-30%, o/(m+n+o+p) being 40%-70%, p/(m+n+o+p) being 10%-25% 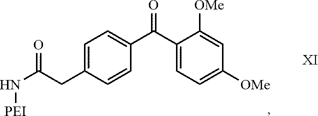 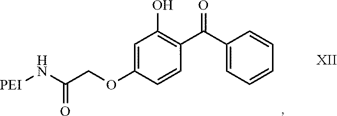 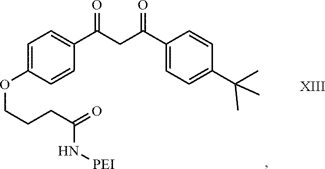
and 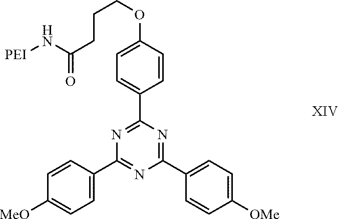
.

11. (canceled)

12. (canceled)

13. The method of claim 1 further comprising a step of mixing the compound A covalently conjugated with the excipient B with an amount of the excipient B polymer that is not covalently bonded with the compound A.

14. The method of claim 1 wherein the compound A conjugated with an excipient B via a covalent bond has one of the following formulae: 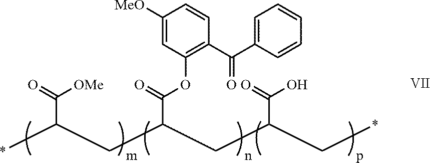 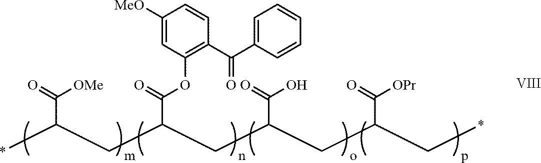 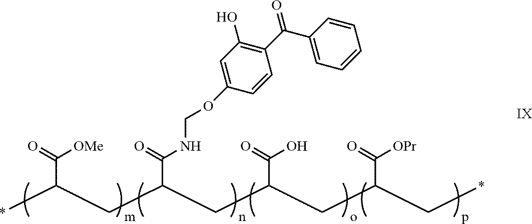 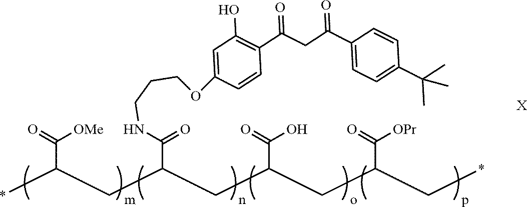
wherein the polymer unit in each of the formulae is a random copolymer unit of three or four monomers, each of m, n, o, and p is the molar number of the corresponding monomer unit, m/(m+n+o+p) is 10%-25%, n/(m+n+o+p) is 2%-30%, o/(m+n+o+p) is 40%-70%, p/(m+n+o+p) is 10%-25%. 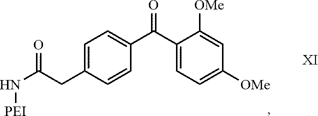 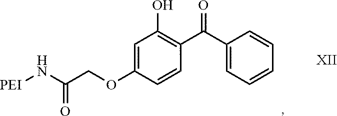 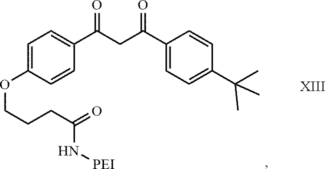
and 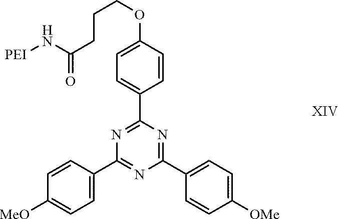
.

15. The method of claim 1 wherein the carrier is an aqueous carrier.

16. The method of claim 1 wherein the covalent bond is the amide bond.

17. The method of claim 1 wherein the polymers and oligomers are water-soluble polymers and oligomers, and each of the polymers and oligomers has a molecular weight of 500-50,000.

18. The method of claim 1 wherein the formulation is homogeneously transparent.

19. The method of claim 1 wherein each of the polymers and oligomers has a molecular weight of 1000-5000.

20. The method of claim 1 wherein the derivatized sunscreen compound is 4-{ 4-[3-(4-tert-butyl-phenyl)-3-oxo-propionyl]-phenoxy}-butyric acid of the following formula (II): 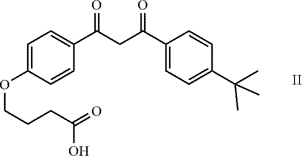 [SMILES: CC(C)(C)c1ccc(C(=O)CC(=O)c2ccc(OCCCC(=O)O)cc2)cc1]
or
4-{4-[3-(4-tert-Butyl-phenyl)-3-oxo-propionyl]-phenoxy}-butyric acid of the following formula (III): 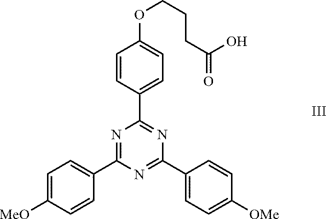 [SMILES: COc1ccc(-c2nc(-c3ccc(OC)cc3)nc(-c3ccc(OCCCC(=O)O)cc3)n2)cc1]
.

21. The method of claim 1 wherein the derivatized sunscreen compound is 1-[4-(3-amino-propoxy)-2-hydroxy-phenyl]-3-(4-tert-butyl-phenyl)-propane-1,3-dione of the following formula (V): 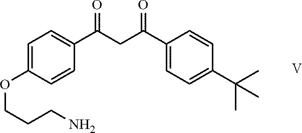 [SMILES: CC(C)(C)c1ccc(C(=O)CC(=O)c2ccc(OCCCN)cc2)cc1]
or
(4-aminomethoxy-2-hydroxy-phenyl) phenyl-methanone of the following formula (VI): 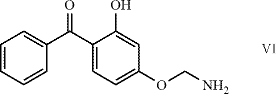 [SMILES: NCOc1ccc(C(=O)c2ccccc2)c(O)c1]
.

22. The method of claim 1 wherein the reacting step comprises reacting the compound A and the excipient B in amounts that result in one molecule excipient B being bonded with multiple molecules compound A of the same type or different types that block different UV bands via multiple ester or amide covalent bonds.
